(19) United States
(12) Patent Application Publication
(10) Pub. No.: US 2009/0163937 A1
(43) Pub. Date: Jun. 25, 2009

(54) DEVICES AND METHODS FOR ACHIEVING MAGNETIC STAND-OFF OF A TISSUE

(75) Inventors: Ghassan S. KASSAB, Indianapolis, IN (US); Jose A. Navia, SR., Buenos Aires (AR)

Correspondence Address: ICE MILLER LLP, ONE AMERICAN SQUARE, SUITE 3100, INDIANAPOLIS, IN, 46282-0200, US

(73) Assignee: CVDevices, LLC, Zionsville, IN (US)

(21) Appl. No.: 12/395,516

(22) Filed: Feb. 27, 2009

Related U.S. Application Data
(63) Continuation-in-part of application No. 12/307,113.
Application No. 12/307,113 is a continuation-in-part of application No. 11/997,147, filed on Jun. 30, 2008.
(60) Provisional application No. 60/817,423, filed on Jun. 30, 2006.
(60) Provisional application No. 60/703,421, filed on Jul. 29, 2005.

(51) Int. Cl. A61B 17/08

(52) U.S. Cl. 606/151

(57) ABSTRACT

Devices and methods are disclosed for providing chronic tissue support and/or remodeling that does not require the use of sutures or staples. A magnetic device having one to three components and at least one pin to maintain a prescribed distance between the magnetic components when the same are magnetically engaged is described. Such devices and methods may be used on any tissue of a body and may be delivered through laparoscopic and/or endoscopic procedures.

### RELATED APPLICATIONS

This U.S. Utility Patent Application (1) is a continuation-in-part of U.S. patent application Ser. No. 12/307,113, filed Dec. 30, 2008, which is a nationalization of International Application No. PCT/US07/15267, filed Jun. 29, 2007, which claims priority to U.S. Provisional Patent Application Ser. No. 60/817,423, filed Jun. 30, 2006; (2) is a continuation-in-part of U.S. patent application Ser. No. 11/997,147, filed Jun. 30, 2008, which is a nationalization of International Application No. PCT/US06/029424, filed Jul. 28, 2006, which claims priority to U.S. Provisional Patent Application Ser. No. 60/703,421, filed Jul. 29, 2005; and (3) claims priority to International Application No. PCT/US08/55303, filed Feb. 28, 2008. The content of each of the foregoing applications is hereby incorporated by reference in its entirety into this disclosure.

### BACKGROUND

Organ and tissue remodeling are clinical techniques that may be applied to numerous different body tissues, ranging from blood vessels to whole organs. Conventionally, such remodeling techniques require incisions and/or sutures in the tissue to be remodeled in order to alter the tissue's anatomy. For example, gastric remodeling is often employed to treat obesity and typically involves the reorganization of the digestive tract. Conventional examples of such procedures involve attempts to either 1) restricting food intake into the body via a restrictive bariatric procedure (a “Restrictive Procedure”), or 2) altering the anatomy of the small intestine or divert the peristalsis of a person's normal food intake past the small intestine to decrease caloric absorption via a malabsorptive bariatric procedure, which is commonly known as a gastric bypass (a “Malabsorptive Procedure”). It is also known to combine the two procedures such that both of the aforementioned techniques are employed jointly.

Malabsorptive Procedures entail an intestinal bypass that results in the exclusion of almost all of the small intestine from the digestive tract. In most Malabsorptive Procedures, a portion of the stomach or small intestine is removed from the digestive tract through a surgical procedure that requires cutting the digestive tissue and thereafter closing any holes or securing the newly formed anatomy with staples and/or sutures. Conversely, Restrictive Procedures generally involve the creation of a passageway extending from the upper portion of the stomach to the lower portion of the stomach in order to decrease the size of the organ and thus prevent the stomach from storing large amounts of food. Conventional Restrictive Procedures rely on the banding and/or stapling of the stomach to create a small pouch on the superior portion of the stomach near the gastroesophageal junction.

Combined operations consisting of Malabsorptive and Restrictive Procedures are the most common bariatric procedures performed today. An example of a combined procedure is the Extended (Distal) Roux-en-Y Gastric Bypass in which a stapling creates a small (approximately 15 to 20 cc) stomach pouch completely separated from the remainder of the stomach. In addition, the small intestine is divided just beyond the duodenum (the hollow tube connecting the stomach to the jejunum), re-arranged into a Y-configuration, and sutured to the small upper stomach pouch to enable the outflow of food therefrom through the newly formed “Roux limb.”

Accordingly, most digestive tract remodeling procedures require that the stomach and/or tissue of the intestine is cut and thereafter sutured or stapled back together. As the digestive tact contains numerous enzymes, strong acids and multiple species of bacteria that assist with digestion, any perforation of a digestive organ is particularly problematic due to the likelihood of leakage therefrom and/or serious infection. As such, these procedures are typically difficult to perform correctly, have high rates of catastrophic post-operative complications that may require prolonged hospitalization and even additional operations, and are often irreversible and/or permanently affect the remodeled tissue and/or organ. Accordingly, a need exists for safe and effective devices and methods for remodeling organs and tissue that are reversible and do not require cutting the underlying tissue and/or the use of sutures or staples.

In addition to remodeling the digestive tract for the treatment of obesity, it is conventionally known to treat various other indications through providing support to the organ or tissue and/or organ or tissue remodeling. For example and without limitation, patients suffering from a symptomatic hiatal hernia may be treated by a Nissen fundoplication where the gastric fundus (the upper portion) of the stomach is wrapped, or plicated, around the inferior part of the esophagus and secured to itself through the use of sutures or staples. In this manner, the gastric fundus of the stomach blocks the enlarged hiatus in the diaphragm and prevents herniation of the stomach therethrough as well as the reflux of gastric acid. As with bariatric surgeries, a Nissen fundoplacation requires that the stomach wall is sutured in order to secure it in position around the esophagus, thereby increasing the risk of complications and preventing the procedure from being easily reversed.

Two laparoscopic surgical techniques exist as alternatives to a Nissen fundoplacation: Tension-Free Techniques and Non-Tension-Free Techniques (referring to the resulting tension—or lack thereof—of the lateral portions of the diaphragm after the procedure). In one example of a Tension-Free Technique, a triangular or semilunar polytef patch is positioned to occlude the anterior segment of the hiatus, which is fixed to the diaphragm with staples or stitches. In conjunction, the stomach is fixed to the abdomen and a fundoplication is performed. The same technique is used for the posterior segment of the hiatus. Conversely, in Non-Tension-Free Techniques, the most common method for hiatal closure is the use of simple stitches or a continuous suture to approach the crural of the diaphragm. Teflon® or Dacron® pledgets or a polypropylene strip are conventionally used to avoid the cutting stitches effect. The pillar closure is covered by a long strip of mesh, which is positioned below the diaphragm in order to reduce the risk of dysphagia or erosion by avoiding the encircling of the oesophagus.

Even when hiatal hernia surgical procedures are a success, the hiatal repair often subsequently fails due to tissue tension. The hiatal crus is a fleshy structure lacking tendinous reinforcement and the use of ordinary sutures to close the hiatal hernia runs a relatively high risk of cutting the muscle. If the hiatus is predominantly wide and the diaphragmatic pillars are necessarily approached with suturing as indicated in many of the above-described techniques, the lateral portions of the diaphragm close to the crura become tense, with probable risk of disruption. Furthermore, in addition to the specific indications discussed herein, there are numerous other conditions for the treatment of which organ and/or tissue remodeling procedures are conventionally employed.

Additionally, it is known to treat various other indications through providing support to an organ or tissue. Abdominal aortic aneurysm is one example of an indication for which conventional techniques of treatment are rather invasive and often require open surgery. An abdominal aortic aneurysm occurs when the large blood vessel that supplies blood to the abdomen, pelvis, and legs becomes abnormally large or balloons outward, thereby forming an aneurysm sac. If left untreated, this weakened area of the aortic wall can progress to aortic dissection or even rupture.

Conventionally, treatment for an abdominal aortic aneurysm involves either open aneurysm repair or endovascular stent grafting. Specifically, traditional open repair involves open abdominal surgery where the abnormal vessel is replaced with a graft made of synthetic material, such as Dacron®. Accordingly, the synthetic graft replaces the weakened area of the aorta and is sutured at its proximal and distal end to the remaining healthy aortic wall. In this manner, the graft allows blood to pass easily therethrough.

Endovascular abdominal aortic aneurysm repair (“EVAR”) is considered an accepted alternative to standard open surgery and avoids major intraabdominal (or retroperitoneal) surgery and the related morbidity and mortality that are associated with standard surgical repair. EVAR is an alternative procedure used in an effort to reinforce or strengthen the weakened aneurysmic area of the aorta that is performed laparoscopically. EVAR typically involves the advancement of a stent graft comprising fabric and metal mesh through the femoral artery and to the afflicted area. Placement of the graft is then achieved such that the graft is positioned within the weakened aortic location of the aneurysm. In this procedure, the proximal and distal ends of the endovascular graft are sutured to healthy portions of the aorta, both proximal and distal to the aortic aneurysm region. Accordingly, the bulge of the aneurysm sac remains; however, the endovascular graft ideally allows blood to flow through the graft and thus bypassing the aneurysm sac.

While EVAR is less invasive than open aneurysm repair, the EVAR procedure typically requires lifelong surveillance by imaging after endograft placement to ensure that the graft continues to function properly. The most common complication associated with EVAR is endoleak. Endoleaks are defined as areas of persistent blood flow outside the lumen of the endograft, either within the aneurysm sac or within connected vascular segments bypassed by the graft. An endoleak following EVAR is considered a failure of the procedure as it is associated with aneurysm enlargement or even rupture. Presence of an endoleak may require additional endovascular interventions or conversion to open repair. Other complications commonly associated with conventional aneurysmic repair procedures include graft migration, thrombosis and/or kinking of the graft. Accordingly, a need exists for safe and effective devices and methods for providing support to weakened or damaged tissue that are noninvasive and reduce or altogether prevent the complications commonly associated with conventionally known support procedures.

It will be appreciated that the foregoing examples were only provided as examples and that there are numerous other indications where intervention is necessary either to remodel the underlying organ or tissue and/or to provide support thereto.

### SUMMARY

Disclosed herein are devices and methods for magnetically engaging a tissue including, but not limited to, reversibly remodeling and/or providing support to a tissue. At least some of the disclosed embodiments provide devices that are capable of being chronically implanted within a body for the purpose of remodeling and/or providing support to a tissue.

In at least one embodiment of an implantable device, the device comprises a first component and a second component. The first component comprises at least two pins extending therefrom and at least one magnet. The second component comprises at least one magnet having a portion that is magnetically biased to attract a portion of the first component. Further, the first and second components are configured to magnetically engage one another through a targeted tissue and the creation of an attractive magnetic force between the first and second components causes the releasable coupling of the at least two pins of the first component with the second component, thereby defining an interior space between the first component and the second component and mechanically engaging the targeted tissue therebetween. The implantable device described herein may be capable of laparoscopic delivery to the targeted tissue.

The at least two pins of the first component may be positioned in various configurations. For example, in at least one embodiment, the first component further comprises a proximal end having at least one pin extending therefrom and a distal end having at least one pin extending therefrom. Additionally, the second component of the implantable device disclosed herein may further comprise at least one receptacle configured to receive at least one of the at least two pins of the first component. In the at least one embodiment of the first component previously described as having at least one pin extending from the proximal end and at least one pin extending from the distal end, the second component of the implantable device may further comprise a proximal end and a distal end, wherein the proximal end of the second component further comprises at least one receptacle configured to receive the at least one pin extending from the proximal end of the first component and the distal end of the second component further comprises at least one receptacle configured to receive the at least one pin extending from the distal end of the first component. In any of the embodiments of the implantable device where the second component comprises at least one receptacle, each of the receptacles may simply be elongated and/or comprise various configurations. For example, each of the at least one receptacles may be configured as an indentation, an elongated indentation, a close-ended hole, or a through-hole. Furthermore, at least one receptacle of the implantable device may comprise a mechanism capable of facilitating the lateral movement of the at least one pin of the first component received therein.

The configurations of the first and second components may be selected depending on the particular patient and/or application of the implantable device. For example and without limitation, the first and second components may each comprise a straight bar configuration, a curved configuration and/or a circular configuration. Furthermore, the first and second components of the implantable device may be flexible or semi-flexible.

In at least one embodiment of the implantable device, the first and second components may each further comprises a first side and a second side. In this at least one embodiment, the magnets of the first and second components are disposed such that the first side of the first component exhibits a magnetic polarity that is opposite of the magnetic polarity of the first side of the second component.

Further, the first component of the implantable device may additionally comprise a channel extending therethrough and a shaft having a proximal end and a distal end. The distal end of the shaft may be configured to be slidably inserted into the channel. Here, in at least one embodiment of the implantable device, all or some of the pins of the first component may be moveable between a retracted position and an extended position. When one of the pins is in the retracted position, the pin is disposed substantially within the channel of the first component and when the pin is in the extended position, the pin extends from the first component. In certain embodiments of the implantable device comprising a channel within the first component, the distal end of the shaft may be configured to apply a force to the pin(s) of the first component and when the distal end of the shaft may be operated to apply the force to at least one of the pins of the first component, the distal end of the shaft causes the at least one pin to move from the substantially retracted position to the extended position.

In addition, the first component may further comprise at least two openings that are in communication with the channel. Here, each of the at least one openings is associated with at least one of the pin(s) of the first component. Furthermore, each of the at least one openings is configured to receive at least one of the pin(s) of the first component therethrough. Each of the pins of the implantable device may comprise a resistance mechanism disposed thereon to bias the pin to the substantially retracted position.

In at least one embodiment of an implantable device, the implantable device may comprise a first component having at least one pin extending therefrom and at least one magnet and a second component comprising at least one pin extending therefrom and at least one magnet having a portion that is magnetically biased to attract a portion of the first component. In this at least one embodiment, the first and second components are configured to magnetically engage one another through a targeted tissue and the creation of an attractive magnetic force between the first component and the second component causes the releasable coupling of the at least one pin of the first component with the second component and the releasable coupling of the at least one pin of the second component with the first component, thereby defining an interior space between the first and second components and mechanically engaging the targeted tissue therebetween. In the at least one embodiment of the implantable device where both the first and second components comprise at least one pin extending therefrom, the first component may further comprise at least one receptacle configured to receive at least one of the at least one pins of the second component and vice versa.

In at least one embodiment of the implantable device, the device comprises a component comprising a first end comprising at least one pin extending therefrom and at least one magnet, a second end comprising at least one magnet having a portion that is magnetically biased to attract a portion of the first end, and a flexible portion disposed in between the first end and the second end and capable of allowing the component to move between a substantially straight configuration and a folded configuration. In this at least one embodiment, the first and second ends are configured to magnetically engage one another through a targeted tissue when the first component is in the folded configuration and the creation of an attractive magnetic force between the first and second ends causes the releasable coupling of the at least one pin of the first end with the second end. In this manner, the implantable device defines an interior space between the first end and the second end and mechanically engages the targeted tissue disposed therebetween. In this at least one embodiment, the second end of the component may further comprise at least one receptacle configured to receive at least one of the at least one pins of the first end of the component. The at least one receptacle may comprise any configuration. For example, each of the at least one receptacles may be elongated in shape and/or comprise an indentation, an elongated indentation, a close-ended hole, or a through-hole. Furthermore, each of the at least one receptacles may further comprise a mechanism capable of facilitating the lateral movement of the at least one pin of the first component received therein. In addition, embodiments of the implantable device may be configured such that it is capable of being laparoscopically delivered to the targeted tissue.

In at least one embodiment of a method for remodeling or providing support to a tissue of interest as described herein, the method comprises the steps of providing an implantable device comprising a first component comprising at least two pins extending therefrom and at least one magnet, and a second component comprising at least one magnet having a portion that is magnetically biased to attract a portion of the first component; the first and second components are configured to magnetically engage one another through a targeted tissue and the creation of an attractive magnetic force between the first component and the second component causes the releasable coupling of the at least two pins of the first component with the second component, thereby defining an interior space between the first component and the second component and mechanically engaging the targeted tissue therebetween; positioning the first component adjacent to a first surface of a tissue of interest; and positioning the portion of the second component that is magnetically biased to attract a portion of the first component adjacent to a second surface of the tissue of interest such that the first component magnetically engages the second component through the tissue of interest, the at least one pin of the first component couple with the second component, and the tissue of interest is disposed therebetween.

Additionally, the method for remodeling or providing support to a tissue of interest may further comprise the step of delivering the implantable device to the tissue of interest laparoscopically. Further, each of the at least two pins of the first component may be capable of moving from a substantially retracted position to an extended position, and at least one embodiment of the methods disclosed herein may further comprise the step of moving the at least two pins of the first component to the extended position.

At least one embodiment of a method for delivering an implantable device to a tissue of interest may comprise the steps of providing an implantable device comprising a first component comprising at least two pins extending therefrom and at least one magnet, and a second component comprising at least one magnet having a portion that is magnetically biased to attract a portion of the first component where the first and second components are configured to magnetically engage one another and the creation of an attractive magnetic force between the first component and the second component causes the releasable coupling of the at least two pins of the first component with the second component, thereby defining an interior space between the first component and the second component and mechanically engaging a targeted tissue therebetween; providing a delivery device for facilitating the laparoscopic delivery of the implantable device, the delivery device comprising a first arm having a proximal end and a distal end, the distal end of the first arm configured to removably couple with the first component, a second arm having a proximal end and a distal end, the second arm capable of rotational movement and the distal end of the second arm configured to removably couple with the second component, a lift system having a proximal end and a distal end, the distal end of the lift system comprising a first branch coupled with the distal end of the first arm and a second branch coupled with the distal end of the second arm, and a hollow casing comprising an elongated tube capable of laparoscopic introduction into a body, the hollow casing having a hollow interior configured to be capable of slidably receiving the implantable device therein, wherein the first arm, the second arm and the lift system are slidably disposed within the hollow interior of the hollow casing such that the first arm is capable of moving independently of the second arm and operation of the second arm causes the first component to become engaged with the second component; inserting the hollow casing laparoscopically into an abdomen; positioning the first component adjacent to a first surface of a tissue of interest through operation of the first arm of the delivery device; and positioning the portion of the second component that is magnetically biased to attract a portion of the first component adjacent to a second surface of the tissue of interest through operation of the second arm of the delivery device such that the first component magnetically engages the second component through the tissue of interest, the at least two pins of the first component couple with the second component, and the tissue of interest is disposed therebetween.

Further, the first arm of the delivery device may further be capable of rotational movement. In method for remodeling or providing support to a tissue of interest may 34. Additionally or alternatively, the distal end of the second arm of the delivery device further comprises a screw-like tip, the second component of the implantable device further comprises a hollow interior configured to receive the screw-like tip of the second arm. Furthermore, the method may further comprise the step of uncoupling the second arm of the delivery device from the second component of the implantable device further comprises the step of unscrewing the screw-like tip of the second arm from the hollow interior of the second component.

In at least one embodiment of the method for remodeling or providing support to a tissue of interest, the method may further comprise the steps of uncoupling the first arm of the delivery device from the first component of the implantable device; uncoupling the second arm of the delivery device from the second component of the implantable device; and withdrawing the delivery device from the body.

In yet another embodiment of the method described herein, at least one of the at least two pins of the first component is moveable between a substantially retracted position and a substantially extended position and the method further comprises the step of moving the at least one moveable pin of the first component to the substantially extended position. In addition, in at least one embodiment, the step of positioning the portion of the second component that is magnetically biased to attract a portion of the first component adjacent to a second surface of the tissue of interest through operation of the second arm of the delivery device further comprises the steps of advancing the second component through the hollow casing; and operating the second arm of the delivery device to rotate the second component such that the portion of the second component that is magnetically biased to attract a portion of the first component magnetically engages the portion of the first component through the tissue of interest.

Kits for performing a medical procedure are further described herein. In at least one embodiment, a kit comprises an implantable device comprising a first component comprising at least two pins extending therefrom and at least one magnet, a second component comprising at least one magnet having a portion that is magnetically biased to attract a portion of the first component, and the first and second components are configured to magnetically engage one another through a targeted tissue and the creation of an attractive magnetic force between the first component and the second component causes the releasable coupling of the at least two pins of the first component with the second component, thereby defining an interior space between the first component and the second component and mechanically engaging the targeted tissue therebetween. In addition, the kit may comprise a fluoroscope or an endoscopic camera.

In at least one alternative embodiment of the implantable device, the implantable device may comprise a first component comprising at least one magnet; a second component comprising at least one magnet; a magnetic graft configured for placement within a vessel lumen, the magnetic graft comprising a proximal end, and a distal end, a body and a hollow interior, wherein both the body and the hollow interior extending between the proximal end and the distal end; and the proximal end of the magnetic graft comprises at least one magnet having a portion that is magnetically biased to attract a portion of the first component through a first targeted tissue and a plurality of pins extending radially therefrom, the distal end of the magnetic graft comprises at least one magnet having a portion that is magnetically biased to attract a portion of the second component through a second targeted tissue and a plurality of pins extending radially therefrom and the creation of an attractive magnetic force between the first component and the proximal end of the magnetic graft causes the releasable coupling of the plurality of pins of the proximal end with the first component and mechanically engages the first targeted tissue therebetween, and the creation of an attractive magnetic force between the second component and the distal end of the magnetic graft causes the releasable coupling of the plurality of pins of the distal end with the second component and mechanically engages the second targeted tissue therebetween.

Further, the vessel lumen of the at least one embodiment of the implantable device described above may comprise an abdominal aorta, the first targeted tissue may comprise a portion of the aorta proximal to an aortic aneurysm, and the second targeted tissue may comprise a portion of the aorta distal to an aortic aneurysm. Here, the first and second components may optionally be capable of laparoscopic or endoscopic delivery to the first and second targeted tissues, respectively.

The first and second components of the implantable device may be configured as previously described herein or in a C-shaped configuration or a ring-shaped configuration. In addition, the first and second components may be flexible or semi-flexible material. Still further, the first and second components each further comprise a joint.

In at least one embodiment of an implantable device, the implantable device comprises a first component comprising at least one magnet and a plurality of pins extending therefrom; a second component comprising at least one magnet and a plurality of pins extending therefrom; and a magnetic graft configured for placement within a vessel lumen, the magnetic graft comprising a proximal end, a distal end, a body and a hollow interior, both the body and the hollow interior extending between the proximal end and the distal end. In addition, the proximal end of the magnetic graft comprises at least one magnet having a portion that is magnetically biased to attract a portion of the first component through a first targeted tissue, the distal end of the magnetic graft comprises at least one magnet having a portion that is magnetically biased to attract a portion of the second component through a second targeted tissue, and the creation of an attractive magnetic force between the first component and the proximal end of the magnetic graft causes the releasable coupling of the plurality of pins of the first component with the proximal end of the magnetic graft and mechanically engages the first targeted tissue therebetween, and the creation of an attractive magnetic force between the second component and the distal end of the magnetic graft causes the releasable coupling of the plurality of pins of the second component with the distal end of the magnetic graft and mechanically engages the second targeted tissue therebetween.

### BRIEF DESCRIPTION OF THE DRAWINGS

FIG. 1A shows a side view of one embodiment of the remodeling device for remodeling and/or supporting a tissue or organ.

FIG. 1B shows a top view of the first and second components of at least one embodiment of the remodeling device shown in FIG. 1A.

FIG. 1C shows the remodeling device of FIG. 1A magnetically engaged with a layer of targeted tissue disposed therebetween.

FIG. 2A shows a side view of the remodeling device of FIG. 1A positioned on a stomach in such a manner so as to create two gastric pouches.

FIG. 2B shows a cross-sectional view taken along line A-A of FIG. 2A.

FIG. 3 shows at least one embodiment of the remodeling device of FIG. 1A.

FIG. 4 shows a side view of at least one embodiment of the remodeling device of FIG. 1A where the first and second components thereof are magnetically engaged with each other.

FIG. 5A shows a view of the first side of at least one embodiment of the second component of the remodeling device 10 shown in FIG. 1A.

FIG. 5B shows a side view of a receptacle of the second component shown in FIG. 5A.

FIGS. 6A and 6B show side views of a remodeling device for remodeling and/or supporting a tissue or organ having a single component and a single pin.

FIGS. 7A and 7B show side views of a remodeling device for remodeling and/or supporting a tissue or organ having a plurality of moveable pins.

FIGS. 8A-8E show embodiments of a remodeling device for remodeling and/or supporting a tissue or organ as applied to treat an abdominal aortic aneurysm.

FIG. 9 shows a flow chart of a method for delivering the remodeling device of FIGS. 8A-8E to a targeted tissue in order to remodel the same or supply support thereto.

FIGS. 10A-10C show side views of a clamp device for assisting with the laparoscopic delivery of the remodeling device of FIG. 1A to a targeted tissue or organ.

FIG. 11 shows at least one embodiment of the clamp device of FIGS. 10A-10C coupled with the second component of at least one embodiment of the remodeling device of FIG. 1A.

FIG. 12 shows a flow chart of a method for laparoscopically delivering the remodeling device of FIG. 1A to a targeted tissue or organ in order to remodel the same or supply support thereto.

FIG. 13 shows a flow chart of a method for laparoscopically delivering embodiments of the remodeling device disclosed herein through the use of the clamp device of FIGS. 10A-10C.

### DETAILED DESCRIPTION

Reference will now be made to the embodiments illustrated in the drawings and specific language will be used to describe the same. It will nevertheless be understood that no limitation of scope is intended by the description of these embodiments.

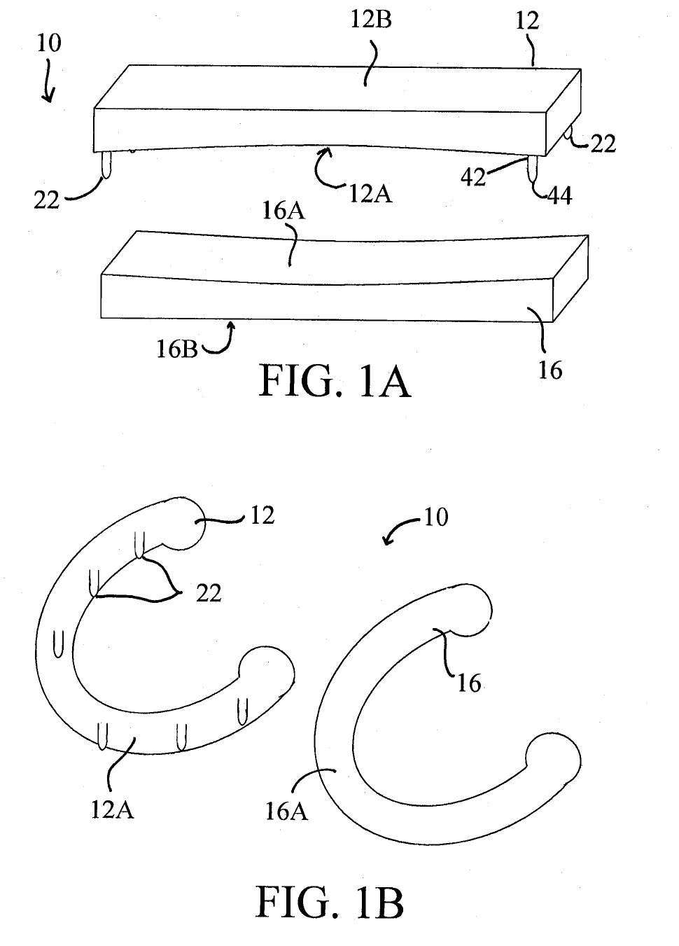

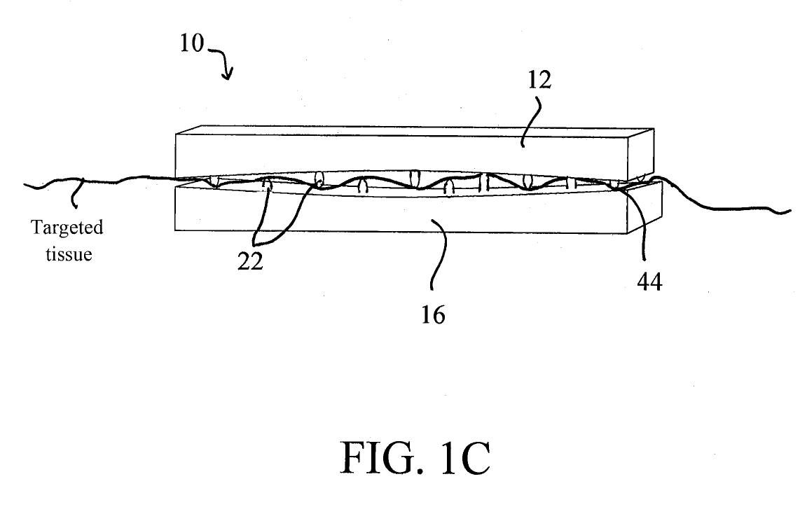

FIGS. 1A, 1B and 1C show schematic views of a remodeling device 10 for remodeling a tissue or organ. In this embodiment, the remodeling device 10 comprises an implantable device and does not require sutures or staples that could lead to dehiscence (e.g., the opening of the suture site), fistula (e.g., an abnormal connection between organs or tissue), or other complications. In addition, while the remodeling device 10 is available for chronic placement within a patient's body, remodeling procedures performed through the use of the device 10 are reversible through minimally invasive procedures.

Now referring to FIG. 1A, the remodeling device 10 is comprised of a first component 12 and a second component 16. The first component 12 comprises a first shape and the second component 16 comprises a second shape that matches at least a portion of the first shape of the first component 12. For example, and without limitation, the first and second components 12, 16 may be configured in a straight bar configuration as shown in FIG. 1A. Alternatively, the first and second components 12, 16 may be configured in a curved, circular, or other configuration (see FIG. 1B). Still further, the first and second components 12, 16 may be configured such that each of the components 12, 16 defines an interior and/or comprise a section of mesh disposed across a portion of such interior as described in detail in U.S. patent application Ser. No. 12/307,113, filed Dec. 30, 2008 and International Application Number PCT/US07/15267, filed Jun. 29, 2007, which are both incorporated by reference herein. It will be understood that the first and second components 12, 16 of the remodeling device 10 may be configured in any shape and may be flexible, semi-flexible, or articulated. Further, a clinician may select a particular configuration of the components 12, 16 of the remodeling device 10 to ensure that the remodeling device 10 appropriately conforms to the tissue or organ of interest.

The first component 12 comprises a proximal end 13, a body having a first side 12A and a second side 12B, and a distal end 14. The first side 12A of the first component 12 is configured to be positioned adjacent to or in contact with a tissue or organ of interest. Likewise, the second component 16 comprises a proximal end 17, a body having a first side 16A and a second side 16B, and a distal end 18. The first side 16A of the second component 16 is configured to be positioned adjacent to or in contact with the tissue or organ of interest.

The first component 12 and the second component 16 each comprise a material suitable to resist corrosion, such as and without limitation, polyurethane, polytetrafluoroethylene (“PTFE”), silastic, titanium, or any other material suitable for use in the medical arts that is corrosion resistant. In this manner, the remodeling device 10 can withstand chronic placement within a body without the risk of deterioration. In addition, the first and second components 12, 16 each comprise one or more magnets. Each of the one or more magnets may comprise any ferromagnetic material known in the art and is capable of magnetically engaging a magnet having an attractive polarity through a tissue.

It will be appreciated that the one or more magnets of each component 12, 16 may comprise either a large portion of the respective component 12, 16 or be disposed within or on a smaller portion of the component 12, 16. Accordingly, each of the at least one magnets may be configured in or on the first and second components 12, 16 in any fashion so long as an attractive magnetic force can be generated between the first side 12A of the first component 12 and the first side 16A of the second component 16. For example, in at least one embodiment, the first component 12 is configured such that the portion of its at least one magnet positioned along the first side 12A of the first component 12 comprises a polarity that is opposite of, and therefore attractive to, the polarity of the portion of the at least one magnet positioned along the first side 16A of the second component 16. Further, in at least one embodiment, the at least one magnet of the first component 12 and/or the at least one magnet of the second component 16 may be encased within a non-corrosive material of the first and second components 12, 16.

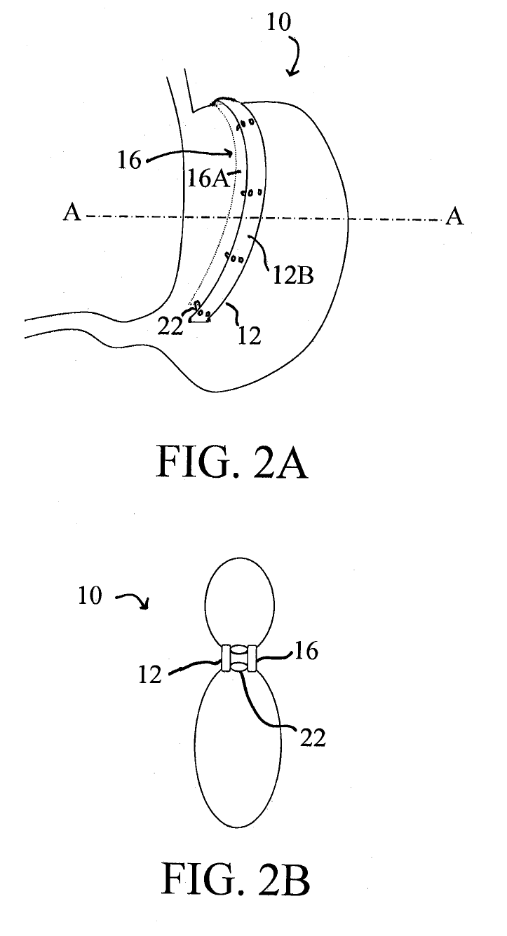

By way of example, in at least one embodiment, the remodeling device 10 may be applied to a stomach for use in dividing the stomach cavity into two pouches such that the effective volume of the stomach is decreased. In this example, as illustrated in FIGS. 2A and 2B, the first side 12A of the first component 12 is applied to the anterior wall of the stomach and the first side 16A of the second component 16 is applied to the posterior wall of the stomach. In this manner, when the magnets of the first side 12A of the first component 12 and the first side 16A of the second component 16 magnetically engage, the stomach tissue disposed therebetween is compressed together, thereby creating two stomach pouches without the use of sutures or staples or perforating the digestive tract.

Due to general magnetic principles (i.e. the two different ends of a magnet exhibit opposite polarities), the portion of the magnets positioned adjacent to the second sides 12B, 16B of the first and second components 12, 16 necessarily comprise a polarity opposite of the polarity of the same magnet at a location adjacent to the first side 12A, 16A, respectively. In this manner, the portion of the at least one magnet positioned adjacent to or along the second side 16B of the second component 16 comprises the same polarity as the portion of the at least one magnet positioned adjacent to the first side 12A of the first component 12. Because like polarities create a repellant force when disposed adjacent to one another, when the second side 16B of the second component 16 is positioned adjacent to the first side 12A of the first component 12, the magnetic portions having like polarities repel one another. This repellant force can be exploited during the delivery of the remodeling device 10 to the organ or tissue of interest, as will be described in further detail herein.

The remodeling device 10 further comprises a plurality of pins 22. In at least one embodiment, the pins 22 extend from both the first component 12 and the second component 16 such that the pins 22 mechanically engage the opposite component when the first and second components 12, 16 are in close proximity and magnetically engaged (see FIG. 1C). In at least one other embodiment, the pins 22 only extend from the first component 12, as shown in FIG. 1A.

Each of the plurality of pins 22 is comprised of a rigid material that does not substantially interfere with the magnetic engagement between the first component 12 and the second component 16. In at least one embodiment, the pins 22 are comprised of a material suitable to resist corrosion, such as and without limitation, polyurethane, PTFE, silastic, titanium, or any other material suitable for use in the medical arts that is corrosion resistant. In addition, the pins 22 may or may not be comprised of a magnetic material as, due to the low amount of surface area in communication with the component 12, 16 with which the pin 22 is engaged, the magnetic properties of the pins 22 are not significant enough to effect the underlying magnetic engagement between the first and second components 12, 16.

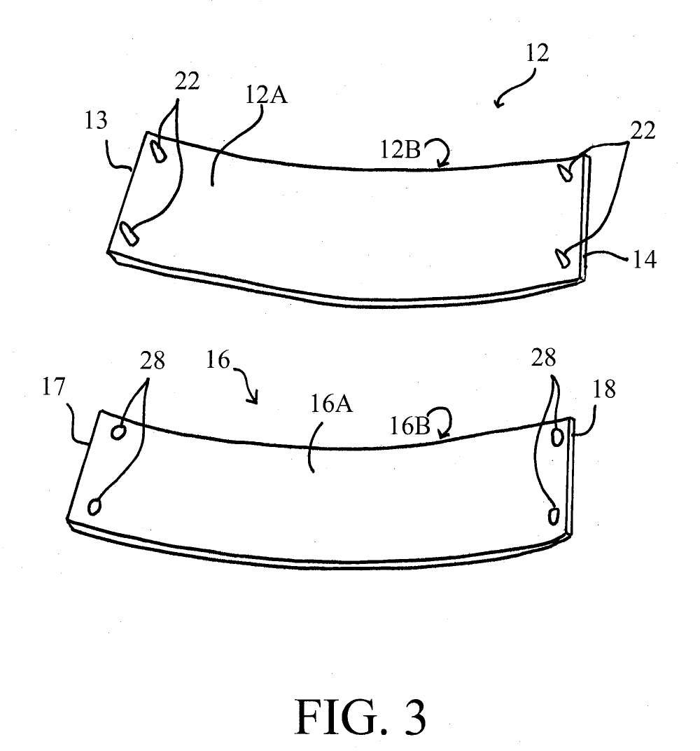

As illustrated in FIGS. 1A and 1C, each of the plurality of pins 22 comprises a proximal end 42 and a distal end 44. The proximal end 42 and the distal end 44 of each pin 22 may be configured similarly or differently, in either a blunt or tapered configuration. In at least one embodiment, each of the pins 22 is metallic and comprises a proximal end 42 that is fixedly coupled with the first side 12A of the first component 12 and a distal end 44 having a tapered configuration. In at least one alternative embodiment, the distal end 44 of each of the pins 22 comprises a blunt configuration. It will be appreciated that any number of pins 22 may be employed in connection with the remodeling device 10. For example, and without limitation, as shown in FIG. 3 the first component 12 of the remodeling device 10 may comprise four pins 22: two pins 22 coupled with the first side 12A on the proximal end 13 of the first component 12 and two pins 22 coupled with the first side 12A on the distal end 14 of the first component 12. However, it is contemplated that the number of pins 22 of the remodeling device 10 will be determined based on the tissue and application for which the remodeling device 10 is to be used.

The pins 22 may comprise any length so long as the pins 22 are of a sufficient size to move through a laparoscopic port and are capable of holding the first component 12 and the second component 16 a distance apart when the first and second components 12, 16 are magnetically engaged. In at least one embodiment, each of the plurality of pins 22 is about 7 to about 16 millimeters long. As previously noted, when the first side 12A of the first component 12 is magnetically engaged with the first side 16A of the second component 16, the pins 22 function to maintain the first component 12 a target distance from the second component 16.

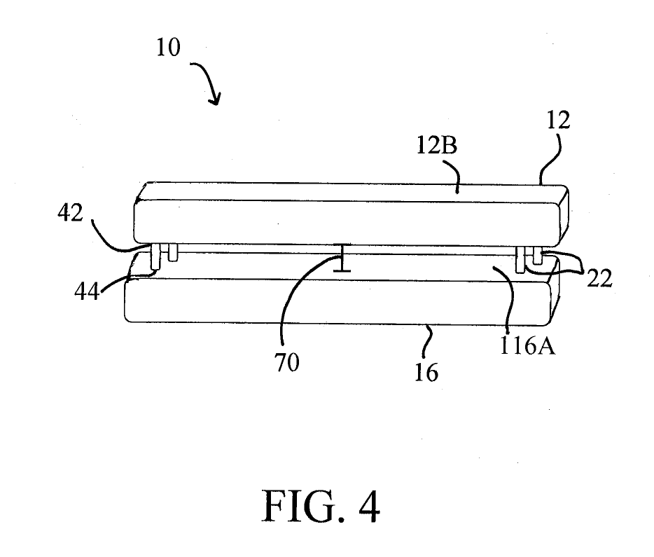

As shown in FIG. 4, when the first sides 12A, 16A of the first and second components 12, 16 are magnetically engaged with one another, the distal ends 44 of the pins 22 are coupled with the first side 16A of the second component 16. In this manner, the pins 22 oppose the magnetic force exerted between the at least one magnet of the first component 12 and the at least one magnet of the second component 16 and prevent the first component 12 from mechanically engaging the second component 16 through any tissue disposed therebetween. Accordingly, an interior space 70 is defined between the first component 12 and the second component 16, the interior space 70 comprising a depth that correlates with the length of the pins 22 of the remodeling device 10. It will be understood that the size of the interior space 70 can be manipulated by the clinician depending on the thickness of the tissue and/or organ to be treated or other factors. For example, to achieve a larger interior space 70, the length of the pins 22 may be increased and/or the thickness of the first and second components 12, 16 may be adjusted. Accordingly, a clinician can easily modify the remodeling device 10 such that it may be optimally configured for a particular application on a particular tissue.

In addition to maintaining an interior space 70 between the components 12, 16 when the components 12, 16 are magnetically engaged with each other, the plurality of pins 22 of the remodeling device 10 further function to secure the remodeling device 10 to the underlying tissue and/or the first component 12 to the second component 12. In other words, the pins 22 prevent the remodeling device 10 from shifting or becoming dislodged from its site of implantation. For example, depending on the configuration of the pins 22 and their arrangement with respect to the first and second components 12, 16, the distal ends 44 of the pins 22 may form a wave-like pattern in the “sandwiched” tissue by pocking the tissue engaged between the first and second components 12, 16. This pattern in the underlying tissue necessarily increases the amount of force required to dislodge the remodeling device 10 from its position on the underlying tissue and/or organ. Accordingly, the pins 22 can provide resistance to the remodeling device 10 shifting or becoming dislodged from its original implantation site on the tissue and/or organ to which it is applied.

To prevent the distal ends 44 of the pins 22 from sliding relative to the first side 16A of the second component 16, in at least one alternative embodiment, the first side 16A comprises a plurality of receptacles 28. Referring back to FIG. 3, the positioning of the receptacles 28 in the first side 16A of the second component 16 correspond with the placement of the plurality of pins 22 on the first component 12. In the at least one embodiment where the first component 12 comprises two pins 22 on the proximal end 13 of the first side 12A and two pins 22 on the distal end of the first side 12A, as shown in FIG. 3, the first side 16A of the second component 16 comprises two receptacles 28 on the proximal end 17, corresponding with the pins 22 on the proximal end 13 of the first component 12, and two receptacles 28 on the distal end 18, corresponding with the pins 22 on the distal end 14 of the first component 12. It will be recognized that any number of receptacles 28 may be disposed in the first side 16A of the second component 16. Furthermore, the first side 12A of the first component 12 may comprise a plurality of receptacles 28 such that, in the at least one embodiment where the second component 16 comprises a plurality of pins 22, the positioning of the receptacles 28 in the first side 12A of the first component 12 correspond with the placement of the plurality of pins 22 extending from the first side 16A of the second component 16.

The receptacles 28 may be configured in any manner so long as each receptacle 28 is capable of receiving the distal end 44 of a pin 22 therein. For example, and without limitation, each of the receptacles 28 may be configured to be an indentation, a closed-ended hole, a through hole, or any configuration suitable for receiving a particular embodiment of the distal end 44 of a pin 22.

By receiving the distal ends 44 of the pins 22 when the first component 12 is magnetically engaged with the second component 16, the receptacles 28 facilitate the secure attachment of the pins 22 with the second component 16 and prevent the pins 22 from sliding or shearing off of the first side 16A. In this manner, the receptacles 28 enhance the overall stability of the remodeling device 10 when the first and second components 12, 16 are magnetically engaged and thus are a safeguard against the migration of the remodeling device 10 when it is applied to a tissue or organ. This is especially advantageous when the remodeling device 10 is applied to a tissue or organ that is susceptible to movement, either through its normal functions or in its remodeled form.

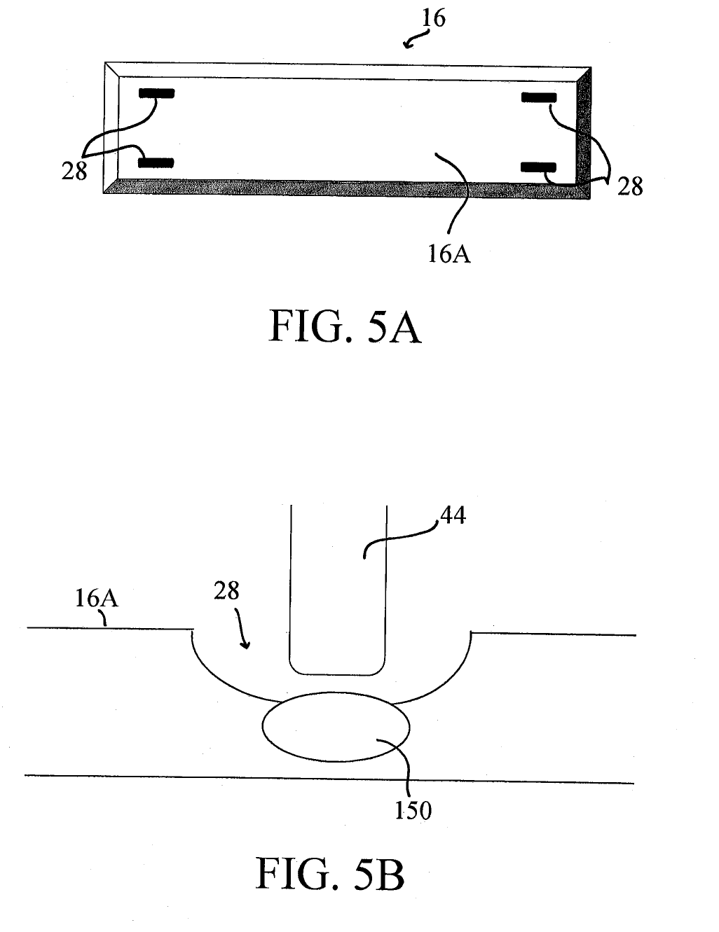

At least one alternative embodiment of the receptacle 28 is shown in FIGS. 5A and 5B. In this embodiment, the receptacles 28 of the second component 16 further comprise a mechanism 150 to allow for lateral movement of the distal end 44 of the pin 22 received within the receptacle 28. The mechanism 150 may comprise any mechanism capable of facilitating the lateral movement of the distal end 44 of a pin 22 within the receptacle 28. In the at least one embodiment shown in FIGS. 5A and 5B, the mechanism 150 comprises a rotating metallic ball disposed within the bottom portion of the receptacle 28. Further, in this at least one embodiment, the receptacle 28 is configured in an elongated shape to allow for movement of the distal end 44 of a pin 22 when the distal end 44 is engaged with the receptacle 28. When this embodiment of the remodeling device 10 is applied to a functioning organ or tissue, the mechanism 150 allows the first component 12 and the second component 16 to shift relative to each other and accommodate the inherent movement of the underlying organ or tissue, thereby preventing the pins 22 from sliding or shearing off of the first side 16A of the second component 16 and potentially breaking the magnetic engagement between the first and second components 12, 16. Accordingly, the shape of the receptacles 28 and the mechanism 150 prevent the remodeling device 10 from migrating relative to its implantation site and substantially decrease the risk that the first and second components 12, 16 will shift or become dislodged from one another and damage the underlying tissue or organ engaged therebetween.

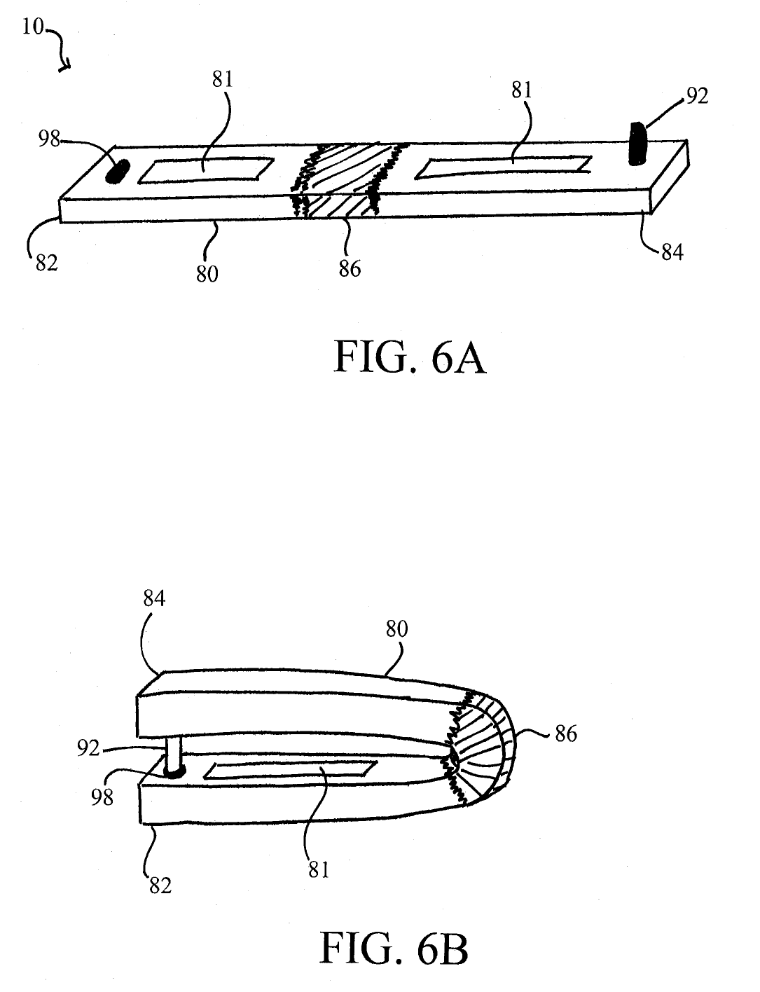

In at least one alternative embodiment of the remodeling device 10 shown in FIGS. 6A and 6B, the remodeling device 10 comprises a single component 80 comprising at least one magnet 81. Furthermore, the remodeling device 10 comprises a first end 82 and a second end 84. The magnet(s) 81 are disposed within or on the single component 80 such that at least a portion of the first end 82 of the single component 80 is magnetically attracted to at least a portion of the second end 84 of the single component 80. For example, and without limitation, in the at least one embodiment shown in FIG. 6A, the single component 80 comprises two magnets 81 disposed thereon. Alternatively, the single component 80 may comprise one magnet 81 that extends substantially the length of the single component 80, or comprise a plurality of magnets 81 disposed in any manner thereon or therein so long as at least a portion of the first end 82 is magnetically attracted to at least a portion of the second end 84 of the magnetic component 80.

In addition, in this at least one embodiment of the remodeling device 10, at least a portion of the single component 80 comprises a flexible or semi-flexible portion 86, such that at least a section of the single component 80 is capable of folding. While the flexible or semi-flexible portion 86 is shown as only a segment of the single component 80 in FIG. 6A, this configuration is offered only by way of offering at least one example. Accordingly, it will be understood that the entire single component 80 may be flexible and/or semi-flexible or any part of the single component 80 may comprise the flexible or semi-flexible portion 86 so long as the single component 80 is capable of folding back on itself such that at least a portion of the first end 82 is capable of magnetically engaging with at least a portion of the second end 84.

In operation, the portion 86 of the single component 80 that is flexible or semi-flexible folds, such that at least a portion of the first end 82 and at least a portion of the second end 84 can magnetically engage each other when the single component 80 is in a folded configuration (see FIG. 6B). In this manner, the at least one embodiment of the remodeling device 10 comprising a single component 80 can function as a clamp. In at least one example, the remodeling device 10 of this at least one embodiment is operable to clamp a portion of the edge of a tissue of interest, thereby providing support thereto and/or a remodeling function as desired.

Similar to the other embodiments of the remodeling device 10 described herein, the at least one embodiment of the remodeling device 10 that comprises a single component 80 also comprises on or more pins 92 and one or more corresponding receptacles 98. In this embodiment, the one or more pin 92 and the one or more corresponding receptacle 98 are both positioned on the single component 80 such that the pin(s) 92 and the receptacle(s) 98 correspond with one another when the single component 80 is in its folded configuration. In this manner, the single component 80 can be used to support or hold underlying tissue without puncturing or overly-compressing the same.

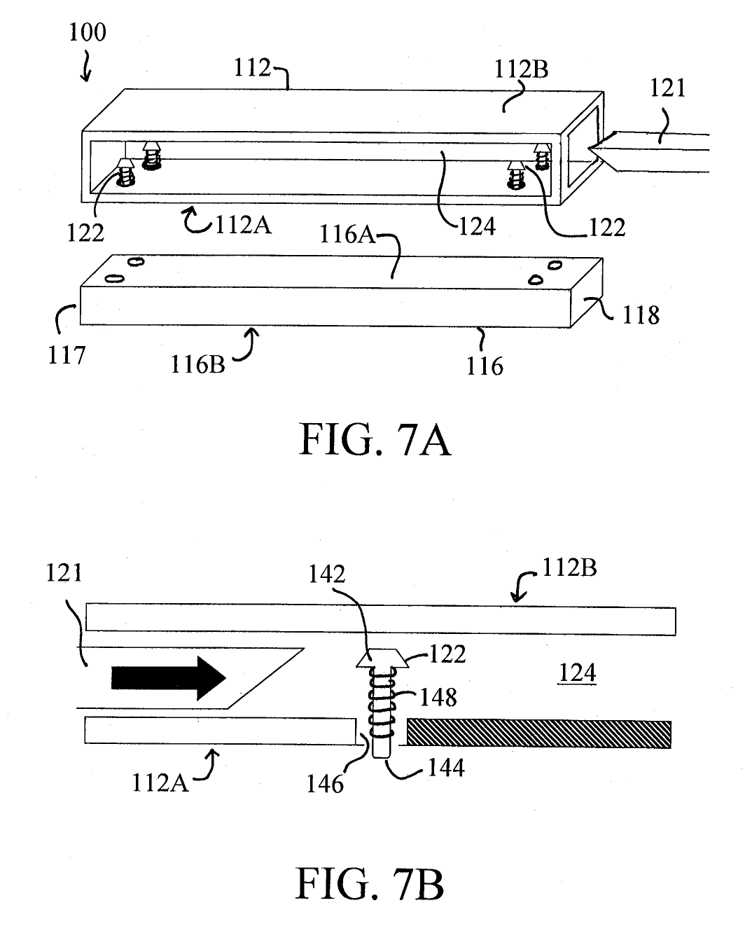

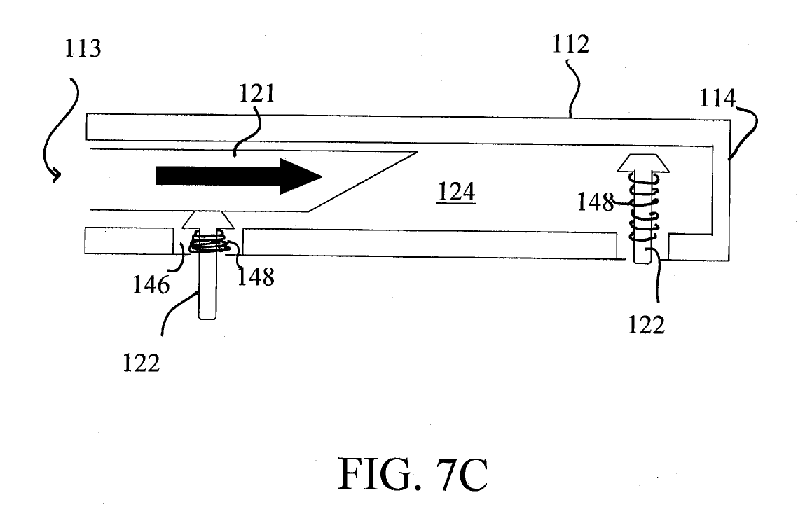

Now referring to FIGS. 7A-7C, at least one additional embodiment of the remodeling device 10 is shown. As shown in FIG. 7A, remodeling device 100 is configured similarly to remodeling device 10; however, remodeling device 100 further comprises a channel 124, a shaft 121, and moveable pins 122. Specifically, remodeling device 100 comprises a first component 112 and a second component 116. Similar to the first component 12 of the remodeling device 10, the first component 112 comprises a proximal end 113, a body having a first side 112A and a second side 112B, and a distal end 114. Furthermore, the first component 112 comprises at least one magnet disposed therein or thereon. Similar to the first side 12A of the first component 12 of the remodeling device 10, the first side 112A of the first component 112 is configured to be positioned adjacent to or in contact with a tissue or organ.

Likewise, the second component 116 of the remodeling device 100 comprises a proximal end 117, a body having a first side 116A and a second side 116B, and a distal end 118. Additionally, the second component 116 also comprises at least one magnet disposed therein or thereon. The first side 116A of the second component 116 is configured to be positioned adjacent to or in contact with a tissue or organ. Each magnet of the first and second components 112, 116 comprises any ferromagnetic material known in the art so long the magnet is capable of magnetically engaging a magnet having an opposite polarity through a tissue. It will be appreciated that the magnets of the remodeling device 100 may comprise either a large portion of the components 112, 116, or is disposed within or on a smaller portion of the components 112, 116. Accordingly, the magnets of the first and second components 112, 116 may be configured in any fashion in or on the first and second components 112, 116 so long as an attractive magnetic force can be generated between the first side 112A of the first component 112 and the first side 116A of the second component 116.

Now referring to FIGS. 7B and 7C, the first component 112 further comprises a channel 124 extending the length thereof, a shaft 121 that is slidably moveable within the channel 124, at least two moveable pins 122 positioned within the channel 124, and at least two openings 146 disposed through the first side 112A. The channel 124 communicates with the proximal end 113 of the first component 112 such that the shaft 121 may be advanced and retracted through the channel 124 through the proximal end 113. In this at least one embodiment, the channel 124 has a depth substantially equal to or greater than the length of the moveable pins 122 and is configured such that the shaft 121 or another similar device may be slidably moved therethrough.

Unlike the pins 22 of the remodeling device 10 which are fixed and thus permanently extend from the first side 12A of the first component 12, the first side 16A of the second component 16, or both, the pins 122 of the remodeling device 100 are movable and disposed within the channel 124 of the first component 112 perpendicular to the longitudinal axis of the channel 124. In each of the locations where a pin 122 is positioned within the channel 124, an opening 146 is disposed through the first side 112A of the first component 112 such that the pin 122 can be extended therethrough (see FIG. 7B). While the second component 116 may alternatively or also comprise a plurality of pins 122 and thus be configured similarly to the first component 112 as described herein, for the sake of simplicity, the majority of the detail for this at least one embodiment is described with respect to the first component 112. Notwithstanding the same, it will be understood that it is within the scope of this disclosure for the second component 116 to comprise the moveable pins 122 rather than the first component 112, or, alternatively, for both the first and second components 112, 116 to comprise the moveable pins 122 and be configured in a similar fashion. The overall configuration of the remodeling device 100 can be determined by the end user based on the patient's specifications, the application for which the remodeling device 100 is to be used, and the particular tissue and/or organ to which the remodeling device 100 will be applied.

It will be appreciated that the pins 122 of the remodeling device 100 are comprised identically to the pins 22 described in connection the remodeling device 10 (excepting that the pins 22 of the remodeling device 10 are fixed and the pins 122 of remodeling device 100 are moveable with respect to the first and/or second components 112, 116). Accordingly, each of the pins 122 of the remodeling device 100 comprises a rigid material such as a metal, a plastic, or any other material suitable for use in the medical arts. Further, as previously stated, each of the pins 122 comprises a proximal end 142 and a distal end 144, and may be of any length so long as the pin 122 is of a sufficient size to move through a laparoscopic port and maintain the first and second components 112, 116 a prescribed distance apart when the same are magnetically engaged through an underlying tissue.

Each of the pins 122 of the remodeling device 100 is capable of moving between a retracted position and an extended position. Further, each of the pins 122 can move independently of the other pins 122 such that one or more of the pins 122 may be in the retracted position while one or more of the pins 122 are in the extended position. When a pin 122 is in the retracted position, the pin 122 is disposed within the channel 124 of the first component 112 such that the pin 122 extends across the width of the channel 124. Depending on the length of the pin 122, the distal end 144 of the pin 122 may or may not protrude through the corresponding opening 146 in the first side 112A of the first component 112 when the pin 122 is in the retracted position. In at least one embodiment, the pins 122 are shorter in length and do not extend past the first side 112A until the pins 122 are moved into the extended position.

As shown in FIG. 7C, when the shaft 121 is advanced through the channel 124 such that a force is applied to the proximal ends 142 of a pin 122, the pin 122 moves from the retracted position to the extended position. Accordingly, the proximal end of the shaft 121 may comprise a pointed configuration to facilitate the application of downward pressure to the proximal ends 142 of the pins 122 when the shaft 121 is advanced there over. As a pin 122 moves into the extended position, the distal end 144 of the pin 126 advances through the respective opening 146 and past the first side 112A of the first component 112.

When the pressure is removed from the distal end 142 of the pin 122 (i.e. the shaft 121 is withdrawn from the channel 124), the pin 122 slidably moves back into the retracted position. Thus, the pin 122 is biased to be positioned in the retracted position. In at least one embodiment of the remodeling device 100, a resistance mechanism 148 is coupled with each of the pins 122 to provide this bias. As shown in FIGS. 7B and 7C, the resistance mechanism 148 comprises a spring system, wherein a spring 148 is coiled around each of the pins 122. In this embodiment, when a pin 122 is positioned in the retracted position, the respective spring 148 is expanded and stores little, if any, potential energy (see FIG. 7B). However, when the pin 122 is moved to the extended position, the spring 148 is compressed and thus stores potential energy (see FIG. 7C). In this manner, the spring 148 provides enough resistance that the pin 122 remains in the retracted position when no pressure is applied. It will be appreciated that any type of resistance mechanism can be employed in connection with the moveable pins 122 of the remodeling device 100 so long as the resistance mechanism is capable of providing resistance to the pins 122 when they are moved to the extended position.

In operation, both remodeling devices 10, 100 may be applied to an organ or tissue of interest in order to remodel the underlying tissue or organ into a desired configuration and/or provide support to the same. As will be discussed in further detail below, the remodeling devices 10, 100 may be used for chronic implantation within a body without the risk of the first and second components migrating through the underlying tissue. Furthermore, because the remodeling devices 10, 100 do not require sutures or staples to achieve remodeling or provide support, implantation of the remodeling devices 10, 100 is entirely reversible and, if desired, the remodeling device 10, 100 may be easily removed from the organ or tissue of interest through a laparoscopic procedure.

As previously described, the specifications of the remodeling devices 10, 100 may be modified to achieve a desired result. For example, and without limitation, the dimensions of the components 12, 16 and/or number of pins 22 may be chosen for a particular application for which the remodeling device 10 is to be used and/or based on the patient. It is also contemplated that the strength of the magnets of the first and second components 12, 16 may also be manipulated to ensure that the proper magnetic strength is employed between the first and second components 12, 16. By manipulating the strength of the at least one magnet comprising both the first and second components 12, 16, a clinician can provide sufficient magnetic attraction such that the remodeling device 10 is securely engaged to the underlying tissue while, at the same time, ensuring that the underlying tissue is not adversely affected by too much compression from the attractive magnetic force generated by the configuration of the remodeling device 10 relative to the organ and/or tissue.

Pursuant to general magnetic principles, magnetic field strength increases with respect to the proximity of the magnet. As such, the closer together the at least one magnet of the first component 12 and the at least one magnet of the second component 16 are positioned, the stronger the attractive magnetic force is between the two components 12, 16. In light of this principle, two magnets placed on opposing sides of a tissue (without any other obstruction positioned therebetween) will migrate through the tissue sandwiched therein, thereby thinning the tissue over time. As the pins 22 impose a limit as to how close the first magnetic component 12 and the second magnetic component 16 can come in relation to one another, the pins 22 effectively prevent the migration of the first and second components 12, 16 when the pins 22 are applied directly to the underlying tissue. By maintaining an interior space 70 between the first and second components 12, 16 when they are magnetically engaged, the pins 22 can prevent the first and second components 12, 16 from overly compressing the tissue, which greatly diminishes, if not eliminates, the risk of the first and second components 12, 16 migrating through the underlying tissue over an extended period of time.

A clinician may select specific permanent magnets to comprise the first and second components 12, 16 of the remodeling device 10 such that the first and second components 12, 16 exert an optimal amount of magnetostatic force to promote the stabilization of the remodeling device 10. For the theoretical application of the remodeling device 10 to reduce the effective volume of a stomach, an example calculation is provided below. In light of the two parallel plates shown in FIG. 1A, the Maxwell's stress tensor is written as follows:

$${T}_{ij} = \frac{1}{μ} [ {B}_{i} {B}_{j} - \frac{1}{2} {B}^{2} {δ}_{ij} ] \qquad [ 1 ]$$

Since only \vec{B}_{z} exists in this application, the Maxwell's stress tensor is written as:

$${T}_{ij} = [ \begin{matrix}- \frac{{\vert {B}_{z} \vert}^{2}}{2 μ} & 0 & 0 \\ 0 & - \frac{{\vert {B}_{z} \vert}^{2}}{2 μ} & 0 \\ 0 & 0 & \frac{{\vert {B}_{z} \vert}^{2}}{2 μ}\end{matrix} ] \qquad [ 2 ]$$

The stress tensor vector which is normal to the surface in two-dimensional coordinates has the form:

$$P = [ \begin{matrix}- \frac{{\vert {B}_{z} \vert}^{2}}{2 μ} & 0 & 0 \\ 0 & - \frac{{\vert {B}_{z} \vert}^{2}}{2 μ} & 0 \\ 0 & 0 & \frac{{\vert {B}_{z} \vert}^{2}}{2 μ}\end{matrix} ] ( \begin{matrix}0 \\ 0 \\ {n}_{z}\end{matrix} ) = \frac{{\vert {B}_{z} \vert}^{2}}{2 μ} \qquad [ 3 ]$$

where, if |B_{z}|=0.5 T, the pressure is calculated as follows:

$$P = \frac{{\vert {B}_{z} \vert}^{2}}{2 μ} = \frac{{0.5}^{2}}{8 π \times {10}^{- 7}} = 99.47 ( kPa ) \qquad [ 4 ]$$

If it is assumed that the angle between the magnetic field B and normal direction of the magnetic plate is taken as 15°, and area=[2π×(1.0×10⁻²)]×(0.5×10⁻²) m², the force is calculated as follows:

F=P×sin 30⁰×area=99.47×0.5×π×0.1=15.62(Newton) [5]

The force determined by Equation 5 represents the tangential force required to oppose or resist movement or migration of the remodeling device 10. Accordingly, the remodeling device 10 can be designed to yield a required force. The area of the remodeling device 10 may also be appropriately designed to spread out the force in order to minimize the compression of the underlying tissue. Other forces may be similarly determined for different geometries and areas under consideration.

In addition to the therapeutic applications of embodiments of the remodeling devices 10, 100 described in U.S. patent application Ser. No. 12/307,113, filed Dec. 30, 2008, and International Application No. PCT/US08/55303, filed Feb. 28, 2008, which are both incorporated herein by reference, additional examples will now be provided with respect to additional applications of embodiments of the remodeling devices 10, 100. While these specific examples refer to particular applications that may be treated through use of the remodeling devices 10, 100, it will nonetheless be understood that the examples described herein are not intended to be limiting and that the remodeling devices 10, 100 may be applied to any tissue or organ of interest. Furthermore, while the remodeling device 10 is described in connection with each of the examples, it will be appreciated that the remodeling device 100 may also be applied to any tissue or organ of interest in a similar manner and use of the remodeling device 10 in lieu of the remodeling device 100, or vice versa, may be determined based on the patient's specifications, the specific application, and/or the tissue or organ in question.

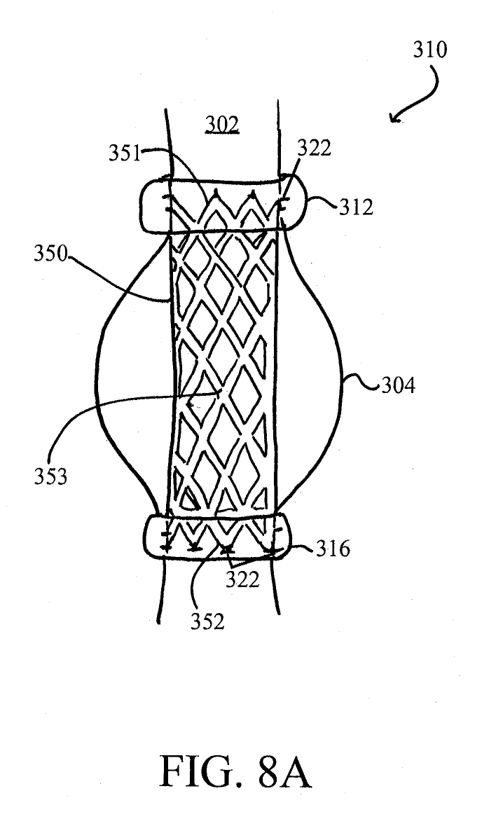

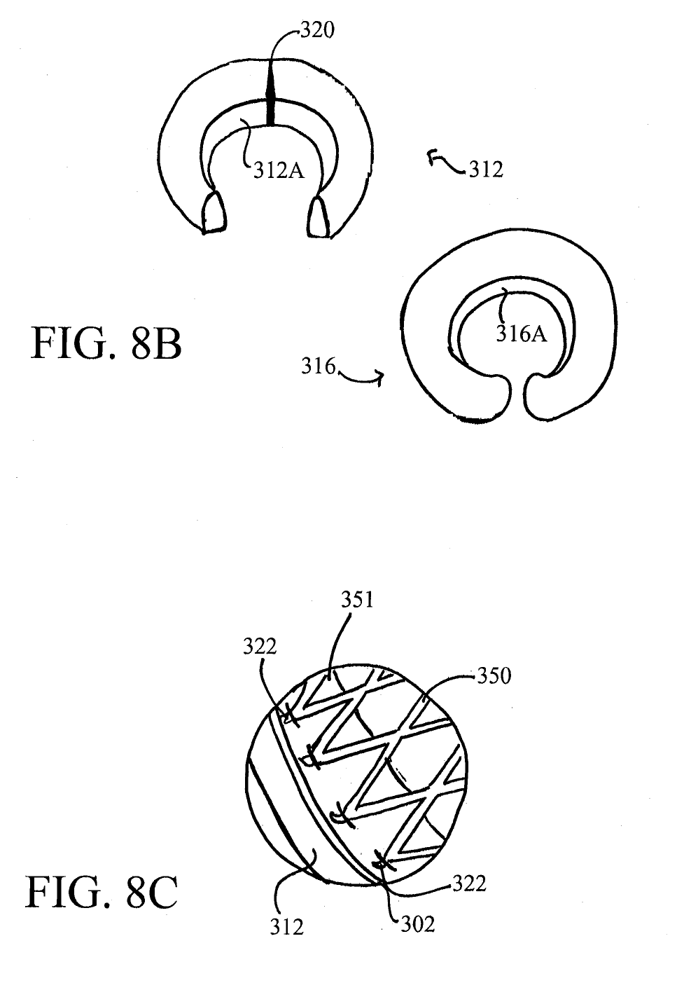

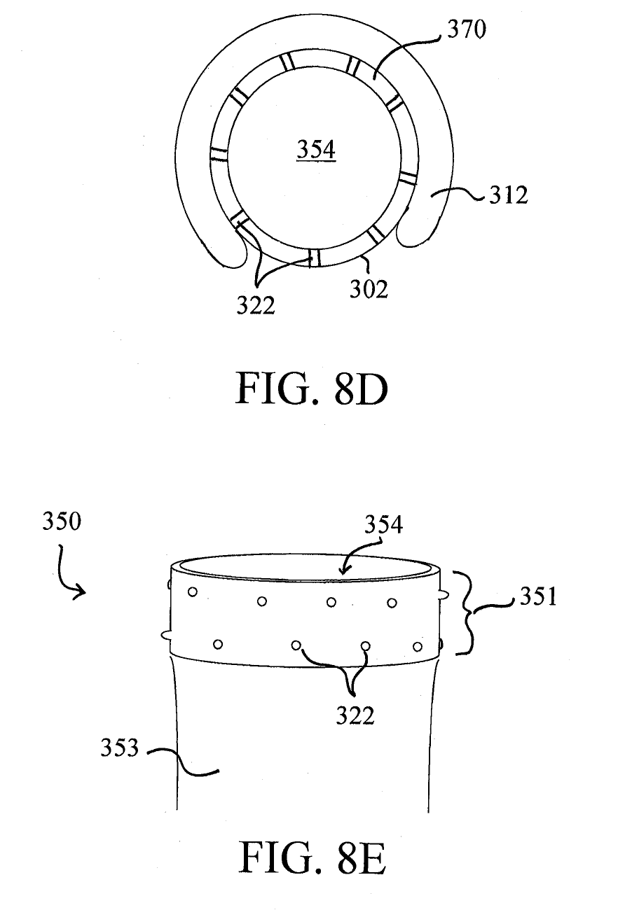

Now referring to FIGS. 8A-8E, at least one additional embodiment of the remodeling device 10 is shown. As illustrated in FIG. 8A, the remodeling device 310 is configured similarly to remodeling device 10; however, the remodeling device 310 further comprises a magnetic graft 350 such that the remodeling device 310 may be used to effectively treat a damaged artery by routing the blood flow through the damaged portion. While specific configurations and examples are described herein, it will be understood that the particular specifications of the remodeling device 310 can be modified on a case-by-case basis depending on the particular patient and application for which the remodeling device 310 is to be used.

FIGS. 8A-8E illustrate a remodeling device 310 as applied to an aorta 302 at the location of an abdominal aortic aneurysm 304. In this manner, the remodeling device 310 may be used as an alternative to open surgical repair, EVAR, or other conventional techniques. As shown in FIG. 8A, components of the remodeling device 310 are used to securely position and stabilize the luminal magnetic graft 350 positioned within the aorta 302. Accordingly, in this at least one embodiment, the first and second components 312, 316 are positioned around the exterior of the aorta 302 and the magnetic graft 350 is delivered endoscopically or otherwise to a location of interest within the aorta 302. Details of this embodiment and the related procedure are described in further detail in U.S. patent application Ser. No. 11/997,147, filed Jun. 30, 2008, which is incorporated by reference herein.

In addition to the magnetic graft 350, the remodeling device 350 comprises a first component 312 and a second component 316. Similar to the first and second components 12, 16 of the remodeling device 10 previously described herein, the first and second components 312, 316 of the remodeling device 350 each comprises a first side 312A, 316A configured to be positioned adjacent to or in contact with a tissue or organ of interest (in this case, the aorta 302). In addition, both the first and second components 312, 316 each comprise at least one magnet disposed therein or thereon. Each magnet of the first and second components 312, 316 comprises any ferromagnetic material known in the art so long as the magnet is capable of magnetically engaging a magnet having an opposite polarity through a tissue.

As shown in FIG. 8A, in this at least one embodiment, the first and second components 312, 316 of the remodeling device 350 comprise a ring- or C-shaped configuration. Accordingly, when the first and second components 312, 316 are applied to the aorta 302, the first and second components 312, 316 may cover only a portion of or the entire circumferential surface of the abdominal aorta 302. In addition, the first and second components 312, 316 may be flexible or semi-flexible such that the components 312, 316 can be easily fitted around the cylindrical exterior wall of the aorta 302. Alternatively, the first and/or second components 312, 316 may comprise a joint 320 that enables the component 312, 316 to easily receive the aorta 302 within the interior of its ring- or C-shaped configuration (see FIG. 8B). Despite the components 312, 316 being depicted as ring- or C-shaped in FIGS. 8A-8E, it will be appreciated that either or both of the first and second components 312, 316 may comprise any other shape (for example and without limitation, staple-shaped, etc.) as long as they are able to produce a sufficient magnetic force when positioned proximal to a magnet having an opposite polarity.

The magnetic graft 350 of the remodeling device 310 comprises an elongated tube configured for placement within a lumen such as the abdominal aorta 302 and has a proximal end 351, a distal end 352, and a body 353 and hollow interior 354 that both extend between the proximal and distal ends 351, 352. The magnetic graft 350 is configured to receive blood flow therethrough and may be comprised of any material commonly used in the medical stenting arts (for example and without limitation, polytetrafluoroethylene, metals, polymers, or fabrics). Further, in at least on embodiment, the body 353 of the magnetic graft 350 may be mesh-like. In the at least one example described herein where the remodeling device 310 is used to treat an abdominal aortic aneurysm, the diameter of the magnetic graft 250 may be configured to have a width that is appropriate for placement within the aortic lumen 302 and the body 353 may be configured to have a length that is slightly longer than the aortic aneurysm 304. In this manner, when the magnetic graft 350 is properly positioned within the aorta 302, the proximal end 351 and the distal end 352 of the magnetic graft 350 are both positioned adjacent to healthy aortic tissue and can physically engage the healthy aortic tissue to secure the magnetic graft 350 in place.

At least a portion of the proximal end 351 of the magnetic graft 350 is capable of magnetically interacting with the first side 312A of the first component 312 of the remodeling device 310 through a tissue. Likewise, at least a portion of the distal end 352 of the magnetic graft 350 is capable of magnetically interacting with the first side 316A of the second component 316 of the remodeling device 310 through a tissue. For example, in at least one embodiment, the proximal and distal ends 351, 352 of the magnetic graft 350 each comprise a ferromagnetic material or powder that is capable of producing a magnetic field when positioned proximal to the first sides 312A, 316A of the first or second components 312, 316. Furthermore, at least the proximal end 351 of the magnetic graft 350 is sized such that it can make physical contact with the inner surface of the aorta 302. Specifically, in at least one embodiment, the proximal end 351 of the magnetic graft 350 is sized to be fitted tightly against the inner surface of the abdominal aorta 302 in order to prevent blood leakage out of the magnetic graft 350 and into the aneurysmic sac 304 via any superfluous space left therebetween. As such tight configuration may make delivery of the magnetic graft 350 to the proper location within the aorta 302 difficult, the magnetic graft 350 may comprise an expandable stent as is known in the art. In this manner, the magnetic graft 350 as a whole may be radially collapsed to facilitate ease of delivery to the desired location and thereafter radially expanded once properly positioned within the desired location of the aorta 302.

Now referring to FIGS. 8C and 8D, in at least one embodiment, both the proximal end 351 and the distal end 352 of the magnetic graft 350 each comprise a plurality of pins 322 extending in a substantially radial direction therefrom. Due to the size of the proximal end 351 of the magnetic graft 350 especially, the plurality of pins 322 may protrude into the inner wall of the aorta 322 and form indentations therein when the magnetic graft 350 is positioned as illustrated in FIG. 8A. This tight fit against the inner wall of the aorta 302, at least with respect to the proximal end 351 of the magnetic graft 350, further reduces the likelihood that any superfluous space may exist in between the aortic wall 302 and the magnetic graft 350. Accordingly, when the remodeling device 310 is properly positioned with respect to the aortic aneurysm 304, the antegrade blood flow through the aorta 302 will be directed into and through the hollow interior 354 of the magnetic graft 350 such that the blood flow bypasses the damaged aneurysmic sac 304.

Similar to the pins 22 described in connection with the remodeling device 10, each of the plurality of pins 322 comprise a rigid material that will not substantially interfere with the magnetic engagement between the first component 312 and the proximal end 351 of the magnetic graft 350 and the second component 316 and the distal end 352 of the magnetic graft 350. In at least one embodiment, the pins 322 are comprised of a material suitable to resist corrosion, such as and without limitation, polyurethane, PTFE, silastic, titanium, or any other material suitable for use in the medical arts that is corrosion resistant. In addition, the pins 322 may or may not comprise a magnetic material and the first and second components 312, 316 may optionally comprise a plurality of receptacles (not shown) positioned to receive the plurality of pins 322 when the first and second components 312, 316 are magnetically coupled with the proximal and distal ends 351, 352, respectively, of the magnetic graft 350.

Similar to the pins 22 of the remodeling device 10, each of the plurality of pins 322 may be configured similarly or differently, in either a blunt or tapered configuration. In at least one embodiment, each of the pins 322 is metallic and comprises a blunt configuration. Further, the pins 322 may comprise any length so long as the pins 322 are of a sufficient size such that, if applicable, the magnetic graft 350 can be delivered endoscopically. In addition, the pins 322 should be long enough to be capable of maintaining the first and second components 312, 316 a sufficient distance away from the proximal and distal ends 351, 352 of the magnetic graft 350, respectively, when such components are magnetically engaged.

It will be appreciated that any number of pins 322 may be employed in connection with the remodeling device 310. For example, as shown in FIG. 8E, the proximal end 351 of the magnetic graft 350 comprises a plurality of pins 322 extending radially therefrom and aligned in two separate rows. In this manner, it can be assured that when the proximal end 351 of the magnetic graft 350 is magnetically engaged with the first component 312 of the remodeling device 310, the two components are coupled in a balanced and stable manner and the first component 312 is unlikely to tilt or disengage from the underlying proximal end 351 of the magnetic graft 350. The distal end 352 of the magnetic graft 350 may further be comprised similarly to the aforementioned description or in any other manner as desired.

It will be understood that any configuration and alignment of pins 322 may be used in connection with the remodeling device 310 and that the aforementioned examples are not intended to be limiting in any manner. For example, it is contemplated that the number of pins 322 of the remodeling device 310 and the layout thereof with respect to the magnetic graft 350 will be determined based on the specific tissue and application for which the remodeling device 310 is to be used.

In at least one alternative embodiment, the first sides 312A, 316A of the first and second components 312, 316 may further comprise a plurality of pins 322 extending therefrom. In this at least one alternative embodiment, the magnetic graft 350 may or may not comprise a plurality of pins 322 and can optionally comprise a plurality of receptacles (not shown) positioned sufficiently to receive each of the plurality of pins 322 of the first and second components 312, 316 when the first and second components 312, 316 are magnetically engaged with the proximal and distal ends 351, 352, respectively, of the magnetic graft 350.

Regardless of which component(s) of the remodeling device 310 comprise(s) the pins 322, the plurality of pins 322 form an interior space 370 between the magnetic graft 350 and the first and second components 312, 316 when the same are magnetically engaged with each other as illustrated in FIGS. 8A and 8D. The length of each of the plurality of pins 322 may be manipulated such that the interior space 370 formed between the first and second components 312, 315 and the magnetic graft 350 maintains a desired area. The interior space 370 created by the plurality of pins 322 of the remodeling device 310 prevents the remodeling device 310 from exerting undue pressure on the underlying tissue sandwiched in between the components of the remodeling device 310 when they are magnetically engaged and allows the remodeling device 310 to provide support to the underlying tissue without causing permanent remodeling or collapse.

In application, the magnetic graft 350 is positioned within the lumen of the aorta 302 (delivered through an endoscopic procedure or otherwise as is known in the art) and the first and second components 312, 316 of the remodeling device 310 are situated adjacent to the external wall of the abdominal aorta 302 or the aneurysmic sac 304 as shown in FIG. 8A. Because the first and second components 312, 316 may cover part of or the entire circumferential surface of the abdominal aorta 302, the first and second components 312, 316 may at least partially ensheathe the abdominal aorta 302 and cover sufficient surface area to interact with the magnetic components of the proximal and distal ends 351, 352, respectively, of the magnetic graft 350 positioned on the inside of the abdominal aorta 302. In this manner, the first and second components 312, 316 can magnetically engage the proximal and distal ends 351, 352 of the magnetic graft 350, respectively. In this non-limiting example, the pins 322 directly engage the inner wall of the aorta 302 and the proximal and distal ends 351, 352 of the magnetic graft 350 are held in position by the magnetic force arising between the first and second components 312, 316 positioned adjacent to the external wall of the aorta 302 and the proximal and distal ends 351, 352 of the magnetic graft 350, respectively.

Accordingly, the plurality of pins 322 assume the majority of the compressional force exerted by the attractive magnets, assist in firmly securing the remodeling device 310 in the proper position, and prevent the remodeling device 310 from overly compressing the underlying aorta 302. Furthermore, the body 353 and hollow interior 354 of the magnetic graft 350 provide a conduit for the blood to flow through the aneurysmic sac 304 such that the blood flow does not contact the aneurysmic sac 304. In this manner, the remodeling device 310 can provide a bypass through the aneurysmic area 304 of the aorta 302 that can be chronically positioned therein without the risk of slippage, migration, or endoleak. In addition, the particular configuration and sizing of the proximal end 351 of the magnetic graft 350 in connection with the restrictive force applied to the external aortic wall 302 by way of the magnetic engagement between the first component 312 with the proximal end 351 of the magnetic graft 350 function to decrease the risk of, and ultimately prevent, endoleak and endotension.

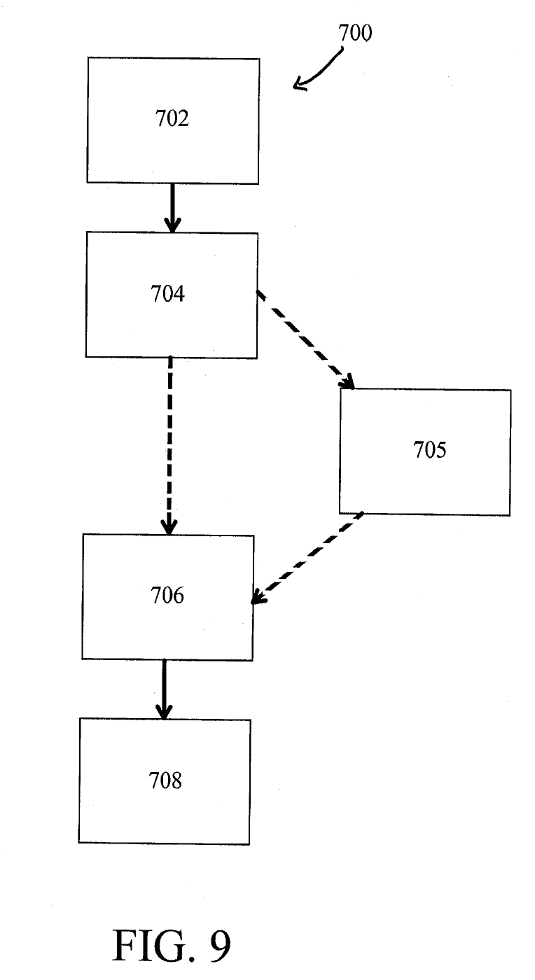

Now referring to FIG. 9, a flow chart of a method 700 for delivering the remodeling device 310 is shown. At step 702, the location of the aneurysmic sac 304 of the aorta 302 is identified and the proper measurements are taken as is known in the art. Thereafter, under fluoroscopic control, direct camera control or otherwise, the proximal end 351 of the magnetic graft 350 is endoscopically advanced from the femoral-iliac artery through the aortic abdominal aneurysm and positioned at a location proximate to the proximate-most region of the aneurysmic sac 304 at step 704. In addition, at this step 704, the clinician should ensure that the distal end 352 of the magnetic graft 350 is properly positioned distally of the distal-most region of the aneurysmic sac 304. Further, if the magnetic graft 350 comprises a collapsible stent and was delivered at step 704 in the collapsed configuration, the magnetic graft 350 may be radially expanded at step 705. If the magnetic graft 350 does not comprise a collapsible stent and/or was delivered in the expanded configuration, the method 700 may proceed directly from step 704 to step 706.

At step 706, under fluoroscopic control, direct camera control or otherwise, the first and second components 312, 316 are advanced laparoscopically into the patient's abdominal cavity and positioned proximate to the tissue of interest. Specifically, in the at least one embodiment where the remodeling device 310 is used to treat an aortic aneurysm, the first component 312 of the remodeling device 310 is positioned at a location proximate to the aneurysmic sac 304 that corresponds with the placement of the proximal end 351 of the magnetic graft 350 within the aorta 302. When the first component 312 of the remodeling device 310 is in the proper location relative to the aorta 302, an attractive magnetic force is created between the first component 312 and the proximal end 351 of the magnetic graft 350, thereby causing the first component 312 and the proximal end 351 to mechanically engage the aortic tissue 302 disposed therebetween. In this manner, the pins 322 of the magnetic graft 350 (or, additionally or alternatively, of the first component 312) bear much of the load of the compression and maintain the interior space 370 between the first component 312 and the proximal end 351 of the magnetic graft 250, within which the aortic wall 302 resides.

Further, at step 708, the second component 316 is positioned at a location distal to the aneurysmic sac 304 that corresponds with the placement of the distal end 352 of the magnetic graft 350 within the aorta 302. Similar to the first component 312 of the remodeling device 310, when the second component 316 is in the proper location, an attractive magnetic force is created between the second component 316 and the distal end 352 of the magnetic graft 350, thereby causing the second component 316 of the remodeling device 310 and the distal end 352 of the magnetic graft 350 to mechanically engage the aortic tissue 302 disposed therebetween. In this manner, the pins 322 of the magnetic graft 322 (or, additionally or alternatively, of the second component 316) bear much of the load of the compression and maintain the interior space 370 between the component 316 and the distal end 352 of the magnetic graft 350, within which the aortic wall resides. It will be understood that steps 706 and 708 may occur simultaneously or in sequence, as may be determined by the particulars of the patient or the preference of the clinician performing the procedure.

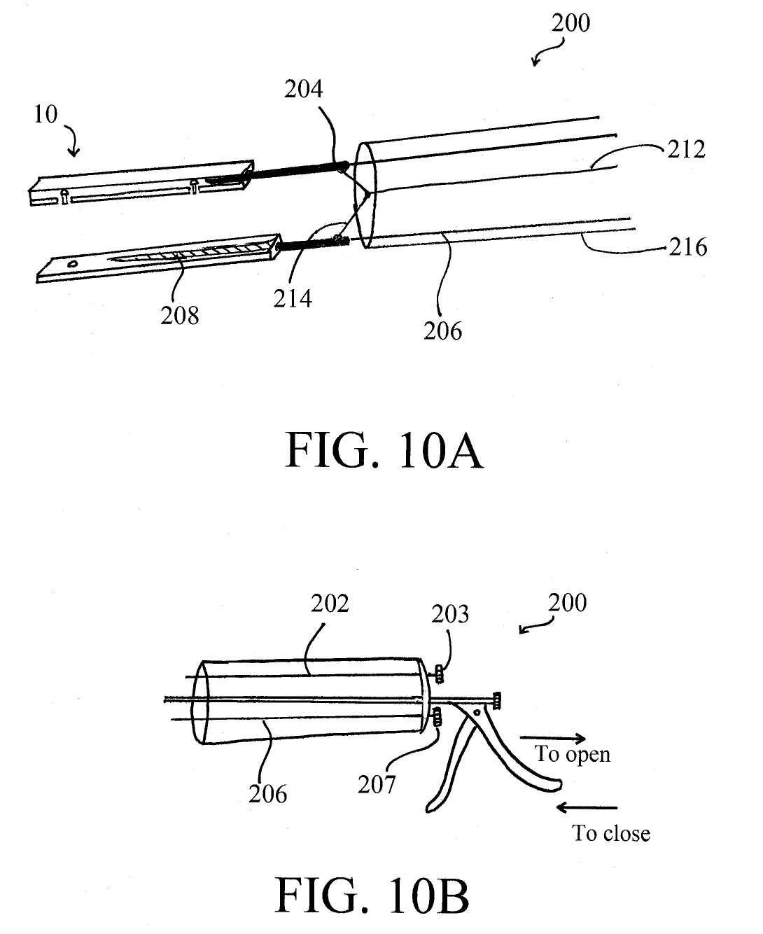

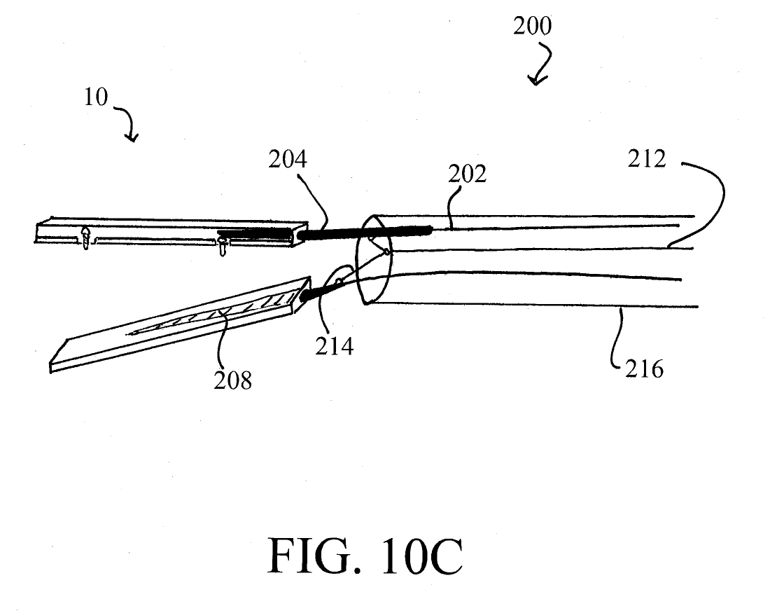

Now referring to FIGS. 10A, 10B and 10C, at least one embodiment of a clamp device 200 is shown. The clamp device 200 may be used to deliver the remodeling device 10 or 100 to the tissue or organ of interest laparoscopically and comprises a first arm 202, a second arm 206 and a lift system 212. Each of the first arm 202, the second arm 206 and the lift system 212 are slidably disposed within a hollow casing 216 configured for laparoscopic delivery. In one embodiment, the hollow casing 216 comprises a distal end for advancement through the body of a patient, and the distal end is open such that the first arm 202, the second arm 206 and the lift system 212 may be delivered therethrough after the distal end of the hollow casing 216 is properly positioned within the body.

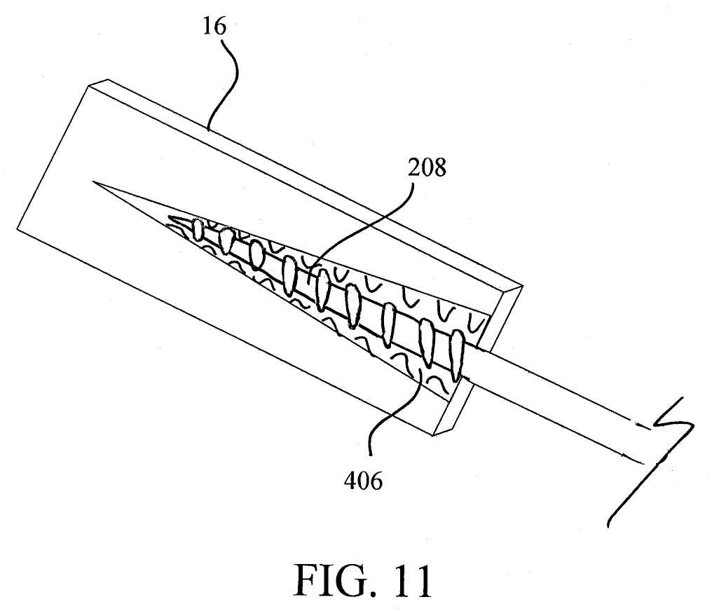

In at least one embodiment, the first arm 202 of the clamp device 200 has a proximal end 203 and a distal end 204 and the second arm 206 of the clamp device 200 has a proximal end 207 and a distal end 208. Further, both the first arm 202 and the second arm 206 may be capable of rotational movement and may comprise any rigid material, such as a metal. In at least one embodiment of the clamp device 200, the distal ends 204, 208 of the first and second arms 202, 206 are both configured in a screw-like configuration. Further, as shown in FIG. 11 at least with respect to the second component 16, the first and second components 12, 16 may each comprise a hollow interior space 402, 406 configured to receive the distal end 204, 208 of either the first or second arms 202, 206 of the clamp device 200. Accordingly, the first arm 202 of the clamp device 200 may be rotatably mated with the interior space 402 of the first component 12, and the second arm 206 may be rotatably mated with the interior space 406 of the second component 16. In this manner, the first and second components 12, 16 may be delivered to the targeted area through use of the clamp device 200 and thereafter easily removed from the arms 202, 206 of the clamp device 200 through rotation of the same.

In at least one alternative embodiment, the first arm 202 of the clamp device 200 may be configured to facilitate the delivery of the first component 112 which comprises moveable pins 122. Specifically, the distal end 204 of the first arm 202 is configured to be removably coupled with the shaft 121 of the first component 112 of the remodeling device 100. Accordingly, in this embodiment of the clamp device 200, only the distal end 208 of the second arm 206 comprises a screw-tip configuration that is capable of rotatably mating with the interior space 406 of the second component 116. It will be appreciated that where the second component 116 comprises one or more moveable pins 122, a channel 124, and a shaft 121, the distal end 208 of the second arm 206 of the clamp device 200 may also, or alternatively, be configured to be removably coupled with the shaft 121 of the second component 116. It will be appreciated that the first arm 202 and the second arm 206 of the clamp device 200 are independent of each other such that a clinician can advance the distal end 204 of the first arm 202 independently of the second arm 206 (and vice versa).

The lift system 212 of the clamp device 200 may be any device capable of moving the first component 12 and the second component 16 relative to each other during the laparoscopic delivery of the remodeling device 10. In at least one embodiment, the lift system 212 comprises a proximal end 213 comprising a hand grip and a distal end. 214 comprising a Y-shaped configuration. Further, the Y-shaped configuration of the distal end 214 comprises a first branch coupled with the distal end 204 of the first arm 202 and a second branch coupled with the distal end 208 of the second arm 206. The branches of the distal end 214 of the lift system 212 are configured such that when no pressure is applied to the hand grip of the proximal end 213, the branches are positioned in an open configuration such that the first and second arms 202, 206 are spaced a distance apart. Likewise, when pressure is applied to the hand grip of the proximal end (i.e. the hand grip is squeezed), the branches of the distal end 214 of the lift system 212 are pulled proximally such that the branches are moved into a closed configuration and the first and second arms 202, 206 are pulled together within the hollow casing 216. Accordingly, moving the branches of the lift system 212 from the open configuration to the closed configuration effectively moves the first and second components 12, 16 relative to one another when the components 12, 16 are coupled with the first and second arms 202, 206 of the clamp device 200. For example, and without limitation, when the branches of the distal end 214 of the lift system 212 are in the open configuration, the first component 12 and the second component 16 are positioned a first distance apart. However, when the branches of the distal end 214 of the lift system 212 are moved to the closed configuration, the first component 12 and the second component 16 are brought together. In this manner, the clamp device 200 can be used to position the first and second components 12, 16 of the remodeling device 10 in the desired location on the targeted organ or tissue.

When the clamp device 200 is employed to deliver the remodeling device 10 to a targeted tissue, the distal ends 204, 208 of the first and second arms 202, 206 are removably coupled with the remodeling device 10, 100. For the sake of simplicity, the clamp device 200 is herein described in connection with delivering the remodeling device 10; however, it will be appreciated that the clamp device 200 may also be utilized to deliver the remodeling device 100. Accordingly, except where expressly stated, any reference herein with respect to use of the clamp device 200 in connection with the remodeling device 10 will be considered to also be applicable to use of the clamp device 200 in connection with the remodeling device 100.

In operation, the remodeling device 10 is positioned within the interior of the hollow casing 216 of the clamp device 200 in preparation for laparoscopic delivery. Due to the magnetic attraction between the first and second components 12, 16 of the remodeling device 10 and the close proximity of the first and second components 12, 16 when they are positioned within the hollow casing 216, in order to facilitate independent delivery of the components 12, 16 to the organ or tissue of interest, it is desirable to prevent the first and second components 12, 16 from magnetically engaging until the device 10 is delivered to the targeted tissue.

As previously described with respect to the configuration of the remodeling device 10, the first side 12A of the first component 12 and the second side 16B of the second component 16 comprise like magnetic polarities and the first side 16A of the second component 16 and the second side 12B of the first component 12 comprise like magnetic polarities. Accordingly, in at least one embodiment, when the remodeling device 10 is positioned within the hollow casing 216, the second arm 206 of the clamp device 200 may be rotated such that the second side 16B of the second component 16 is positioned adjacent to the first side 12A of the first component 12. Alternatively, the first arm 202 of the clamp device 200 may be rotated such that the first component 12 is positioned with its second side 12B adjacent to the first side 16A of the second component 16. As like-polarities generate a repellant force, when so positioned, the two components 12, 16 repel one another, which facilitates their independent maneuverability within the hollow casing 216. After the first and second components 12, 16 are maneuvered out of the hollow casing 216 through use of the first and second arms 202, 206, the user can use the lift system 212 to maneuver the first and second components 12, 16 relative to each other and the targeted tissue and/or organ.

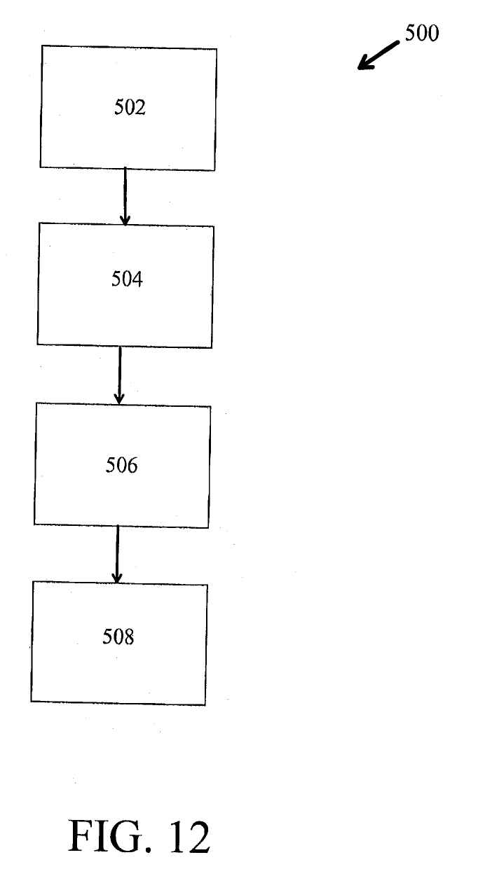

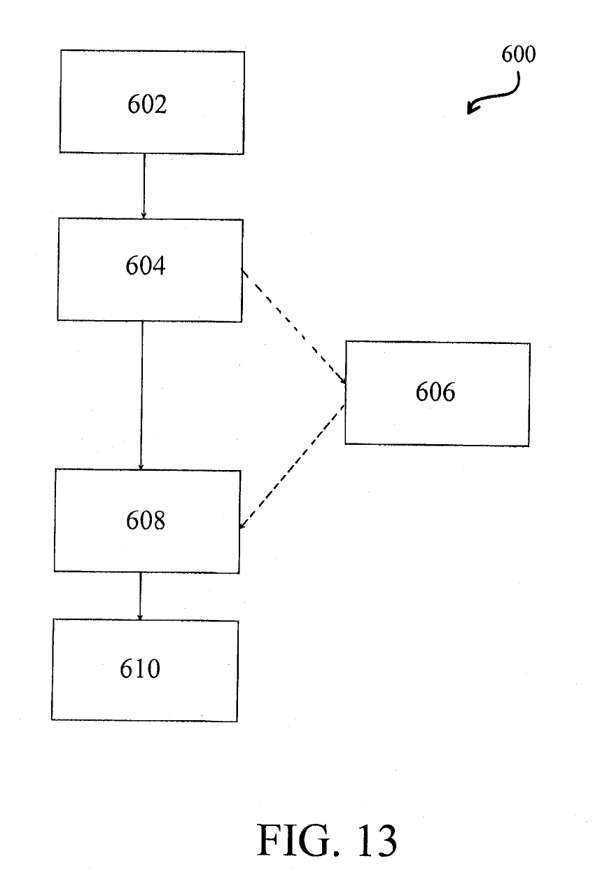

Now referring to FIG. 12, a flow chart of a method 500 for laparoscopically delivering the remodeling device 10 is shown. For ease of understanding, the steps of the related methods described herein will be discussed relative to the components of the remodeling device 10 and, at least in part, the clamp device 200, but it will be appreciated by one skilled in the art that any device can be used to perform these methods so long as the device is capable of magnetically engaging a magnetic composition through a piece of tissue and the resulting magnetic engagement is secure. Furthermore, while the methods described herein are described in connection with embodiments of the remodeling device 10, the remodeling device 100, and/or the clamp device 200, it will be appreciated that various additional devices may be used to achieve the laparoscopic delivery such as a camera and/or a device for delivering a gas to a targeted area.

At step 502, the first and second components 12, 16 are advanced laparoscopically into the patient's body. In the aforementioned embodiment and the at least one embodiment where the remodeling device 10 comprises a single flexible component, the component(s) may be inserted through a catheter into the appropriate cavity of the patient's body. Under fluoroscopic, direct camera control or otherwise, at step 504, the first side 12A of the first component 12 (or in the embodiment where the remodeling device 10 comprises a single component, the first end of the single flexible component) is positioned adjacent to the desired surface of a targeted tissue. Accordingly, in an embodiment where the first side 12A of the first component 12 comprises a plurality of pins 22, the distal end of each of the pins 22 are positioned proximate to the desired surface of the targeted tissue. In addition, at step 506, under fluoroscopic, direct camera control or otherwise, the first side 16A of the second component 16 (or in the embodiment where the remodeling device 10 comprises a single component, the second end of the single flexible component) is positioned on an opposite side of the targeted tissue such that the desired affect may be achieved when the first and second 12, 16 (or the first end and second end of the single flexible component) are magnetically engaged.

Thereafter, the first and second components 12, 16 (or, in the embodiment where the remodeling device 10 comprises a single component, the first and second ends of the single flexible component) magnetically engage at step 508 such that the targeted tissue is sandwiched therebetween. In the at least one embodiment where either or both of the first sides 12A, 16A of the first and second components 12, 16 comprise a plurality of pins 22 extending therefrom, when the first sides 12A, 16A of the first and second components 12, 16 magnetically engage, the distal ends 44 of the pins 22 either contact the underlying targeted tissue and are supported by the opposite component through the tissue, or contact the opposite component of the remodeling device 12, 16 directly. In this manner, the pins 22 take up all of the load and prevent the over-compression of the underlying targeted tissue. Accordingly, depending on how the remodeling device 10 is applied to the targeted tissue, the remodeling device 10 is capable of effectively remodeling and/or providing support to the underlying tissue or organ in a desired manner.

Now referring to FIG. 13, a flow chart of a method 600 for laparoscopically delivering the remodeling device 10 through the use of the clamp device 200 is shown. At step 602, the distal end of the hollow casing 216 is advanced laparoscopically into a patient's body cavity. Under fluoroscopic, direct camera control or otherwise, the distal end of the hollow casing 216 is positioned proximate to the tissue of interest. As previously described, while the remodeling device 10 is positioned within the hollow casing 216, the first side 12A of the first component 12 and the second side 16B of the second component 16 (or vice versa) are positioned adjacent to one another such that the magnetic components of each component 12, 16 repel each other. At step 604, after the distal end of the hollow casing 216 is properly positioned within the patient's body cavity, the first and second arms 202, 206 are advanced through the hollow casing 216, thereby moving the remodeling device 10 through the distal end of the hollow casing 216 and into the body cavity. Accordingly, the first side 12A of the first component 12 is positioned adjacent to one side of the targeted tissue and the second side 16B of the second component 16 is positioned adjacent to the opposite side of the targeted tissue.

After the components 12, 16 are sufficiently positioned relative to the targeted tissue, the method 600 advances to step 608. At step 608, the second arm 206 is rotated 180° such that the first side 16A of the second component 16 is positioned adjacent to the targeted tissue. Accordingly, as the first side 16A of the second component 16 comprises at least one magnet having the opposite polarity of the first side 12A of the first component 12, an attractive magnetic force is created between the two components 12, 16, thereby causing the first component 12 and the second component 16 to move together and mechanically engage the underlying targeted tissue. In this manner, the pins 22 bear much of the load of the compression and maintain an interior area 70 between the components 12, 16, within which the targeted tissue resides.

After the remodeling device 10 is properly positioned on the targeted tissue, the clamp device 200 can be withdrawn from the body cavity at step 610. Specifically, at step 610, the first arm 202 and the second arm 206 of the clamp device 200 are detached from the first and second components 12, 16, respectively. In the at least one embodiment where the first and second components 12, 16 each comprise a hollow interior space 402, 406, respectively, the first arm 202 is detached from the first component 12 by rotating the first arm 202 and unscrewing the distal end 204 from the hollow interior space 402 of the first component 12. Similarly, the second arm 206 is detached from the second component 16 in much of the same manner by rotating the second arm 206 and unscrewing the distal end 208 thereof from the hollow interior space 406 of the second component 16. Thereafter, the clamp device 200 is withdrawn from the body cavity thereby leaving the remodeling device 10 to remain implanted on the targeted tissue.

It will be understood that the remodeling device 100 can be delivered to a targeted tissue using either method 500 or method 600. However, because the pins 122 of the remodeling device 100 are moveable, it is necessary to either deliver the remodeling device 100 with the pins 122 already locked in the extended position, or to deploy the moveable pins 122 after the components 112, 116 of the remodeling device 100 have been properly positioned relative to the targeted tissue. Accordingly, in the event the clinician desires to deliver the remodeling device 100 to the targeted tissue with the pins 122 in the collapsed position and thereafter move the pins 122 into the expanded position, after step 604 of the method 600 and the components 112, 116 are sufficiently positioned relative to the targeted tissue, the pins 122 are moved to the extended position at step 606. Specifically, at step 606, the pins 122 are extended by advancing the first arm 202 of the clamp device 200 distally, which thereby slidably moves the shaft 121 through the channel 124 of the first component 112. In this manner, the shaft 121 applies downward pressure to the proximal ends 142 of the pins 122, which causes the pins 122 to move to the extended position. In at least one embodiment, after the shaft 121 has deployed the pins 122, the shaft 121 may be secured within the channel 124 by a locking mechanism (not shown) such that the pins 122 remain in the extended position. The locking mechanism may comprise a latching mechanism, a clip, a fastener, or any other mechanism that is capable of retaining the shaft 121 within the channel 124. In at least one alternative embodiment, the dimensions of the shaft 121 can be manipulated to affect how far the pins 122 extend from the first side 112A of the first component 112. For example and without limitation, the depth of the shaft 121 can be configured to be less than the depth of the channel 124 such that when the shaft 121 is used to move the pins 122 into the extended position, the pins 122 do not fully extend through the openings 146 and the springs 148 are not fully compressed. Further, it will be appreciated that step 606 may occur prior to step 604 such that the pins 122 of the first component 112 are deployed prior to advancing the first and second arms 202, 206 through the hollow casing 216 and into the body cavity.

The remodeling devices described herein and the clamp device 200 provide numerous benefits over the devices and systems of the prior art. The remodeling device 10, 100, 310 may be inserted laparoscopically and/or endoscopically, is minimally invasive, completely reversible and available for chronic placement without the risk of complications. Furthermore, use of the remodeling device 10, 100, 310 to treat and/or support a targeted tissue or organ produces a reduced amount of negative side effects than the procedures of the prior art for similar indications. In addition, the clamp device 200 allows the remodeling device 10, 100 to be easily delivered in a procedure that takes as little as ten (10) minutes.

While the remodeling devices 10, 100, 310 are presented with respect to specific anatomy and treatment examples, as one of ordinary skill in the art would recognize, the remodeling devices 10, 100, 310 and the methods 500, 600 and 700 may be expanded to any organ, limb or body structure that would benefit from reshaping, remodeling, or added support using reversible, easy to use, and easy to implement techniques for chronic placement.

The devices and methods have been presented in detail with reference to certain embodiments thereof, however, such embodiments are offered by way of non-limiting examples, as other versions are possible. It is anticipated that a variety of other modifications and changes will be apparent to those having ordinary skill in the art and that such modifications and changes are intended to be encompassed within the spirit and scope of the devices and methods as defined by the following claims.

1. An implantable device comprising:
a first component comprising at least two pins extending therefrom and at least one magnet;
a second component comprising at least one magnet having a portion that is magnetically biased to attract a portion of the first component; and
the first and second components are configured to magnetically engage one another through a targeted tissue and the creation of an attractive magnetic force between the first component and the second component causes the releasable coupling of the at least two pins of the first component with the second component, thereby defining an interior space between the first component and the second component and mechanically engaging the targeted tissue therebetween.

2. The implantable device of claim 1, wherein the second component further comprises at least one receptacle configured to receive at least one of the at least two pins of the first component.

3. The implantable device of claim 1, wherein the first and second components are capable of laparoscopic delivery to the targeted tissue.

4. The implantable device of claim 1, wherein the configuration of the first and second components is selected from a group consisting of a straight bar configuration, a curved configuration and a circular configuration.

5. The implantable device of claim 1, wherein the first and second components are flexible or semi-flexible.

6. The implantable device of claim 1, wherein the first component further comprises a first side and a second side and the second component further comprises a first side and a second side, and the magnets of the first and second components are disposed such that the first side of the first component exhibits a magnetic polarity that is opposite of the magnetic polarity of the first side of the second component.

7. The implantable device of claim 1, wherein the first component further comprises a proximal end having at least one pin extending therefrom and a distal end having at least one pin extending therefrom.

8. The implantable device of claim 7, wherein the second component further comprises:
a proximal end comprising at least one receptacle configured to receive the at least one pin extending from the proximal end of the first component; and
a distal end comprising at least one receptacle configured to receive the at least one pin extending from the distal end of the first component.

9. The implantable device of claim 2, wherein the configuration of each of the at least one receptacles is selected from a group consisting of an indentation, an elongated indentation, a closed-ended hole, and a through-hole.

10. The implantable device of claim 2, wherein the at least one receptacle further comprises a configuration that is elongated.

11. The implantation device of claim 2, wherein the at least one receptacle further comprises a mechanism capable of facilitating the lateral movement of the at least one pin of the first component received therein.

12. An implantable device comprising:
a first component comprising at least one pin extending therefrom and at least one magnet;
a second component comprising at least one pin extending therefrom and at least one magnet having a portion that is magnetically biased to attract a portion of the first component; and
the first and second components are configured to magnetically engage one another through a targeted tissue and the creation of an attractive magnetic force between the first component and the second component causes the releasable coupling of the at least one pin of the first component with the second component and the releasable coupling of the at least one pin of the second component with the first component, thereby defining an interior space between the first component and the second component and mechanically engaging the targeted tissue therebetween.

13. The implantation device of claim 12, wherein the first component further comprises at least one receptacle configured to receive at least one of the at least one pins of the second component.

14. The implantation device of claim 1, wherein the first component further comprises a channel extending therethrough and a shaft having a proximal end and a distal end, the distal end of the shaft configured to be slidably inserted into the channel.

15. The implantation device of claim 1, wherein the at least two pins of the first component are moveable.

16. The implantable device of claim 15, wherein the at least two pins of the first component are moveable between a retracted position and an extended position and wherein when one of the at least two pins of the first component is in the retracted position the pin is disposed substantially within the channel of the first component, and when at least one of the at least two pins of the first component is in the extended position, the pin extends from the first component.

17. The implantable device of claim 16, wherein the distal end of the shaft is configured to apply a force to the at least two pins of the first component; and
wherein when the distal end of the shaft is operated to apply the force to at least one of the at least two pins of the first component, the distal end of the shaft causes the at least one pin to move from the substantially retracted position to the extended position.

18. The implantable device of claim 17, wherein the first component further comprises at least two openings that are in communication with the channel, each of the at least one openings associated with at least one of the at least two pins of the first component and configured to receive at least one of the at least two pins of the first component therethrough.

19. The implantable device of claim 18, wherein each of the at least two pins of the first component further comprises a resistance mechanism disposed thereon to bias the pin to the substantially retracted position.

20. An implantable device comprising:
a component comprising a first end comprising at least one pin extending therefrom and at least one magnet, a second end comprising at least one magnet having a portion that is magnetically biased to attract a portion of the first end, and a flexible portion disposed in between the first end and the second end and capable of allowing the component to move between a substantially straight configuration and a folded configuration; and
the first and second ends are configured to magnetically engage one another through a targeted tissue when the first component is in the folded configuration and the creation of an attractive magnetic force between the first end and the second end causes the releasable coupling of the at least one pin of the first end with the second end, thereby defining an interior space between the first end and the second end and mechanically engaging the targeted tissue therebetween.

21. The implantable device of claim 20, wherein the second end of the component further comprises at least one receptacle configured to receive at least one of the at least one pins of the first end of the component.

22. The implantable device of claim 20, wherein the component is capable of laparoscopic delivery to the targeted tissue.

23. The implantable device of claim 21, wherein the configuration of the at least one receptacle is selected from a group consisting of an indentation, an elongated indentation, a closed-ended hole, and a through-hole.

24. The implantable device of claim 21, wherein the at least one receptacle further comprises a configuration that is elongated.

25. The implantation device of claim 21, wherein the at least one receptacle further comprises a mechanism capable of facilitating the lateral movement of the at least one pin of the first component received therein.

26. A method for remodeling or providing support to a tissue of interest, the method comprising the steps of:
providing an implantable device comprising:
a first component comprising at least two pins extending therefrom and at least one magnet,
a second component comprising at least one magnet having a portion that is magnetically biased to attract a portion of the first component, and
the first and second components are configured to magnetically engage one another through a targeted tissue and the creation of an attractive magnetic force between the first component and the second component causes the releasable coupling of the at least two pins of the first component with the second component, thereby defining an interior space between the first component and the second component and mechanically engaging the targeted tissue therebetween;
positioning the first component adjacent to a first surface of a tissue of interest; and
positioning the portion of the second component that is magnetically biased to attract a portion of the first component adjacent to a second surface of the tissue of interest such that the first component magnetically engages the second component through the tissue of interest, the at least one pin of the first component couple with the second component, and the tissue of interest is disposed therebetween.

27. The method of claim 26, wherein each of the at least two pins of the first component is capable of moving from a substantially retracted position to an extended position and further comprising the step of moving the at least two pins of the first component to the extended position.

28. The method of claim 26, further comprising the step of delivering the implantable device to the tissue of interest laparoscopically.

29. A method for delivering an implantable device to a tissue of interest comprising the steps of:
providing an implantable device comprising:
a first component comprising at least two pins extending therefrom and at least one magnet,
a second component comprising at least one magnet having a portion that is magnetically biased to attract a portion of the first component, and
the first and second components are configured to magnetically engage one another and the creation of an attractive magnetic force between the first component and the second component causes the releasable coupling of the at least two pins of the first component with the second component, thereby defining an interior space between the first component and the second component and mechanically engaging a targeted tissue therebetween;
providing a delivery device for facilitating the laparoscopic delivery of the implantable device, the delivery device comprising:
a first arm having a proximal end and a distal end, the distal end of the first arm configured to removably couple with the first component,
a second arm having a proximal end and a distal end, the second arm capable of rotational movement and the distal end of the second arm configured to removably couple with the second component,
a lift system having a proximal end and a distal end, the distal end of the lift system comprising a first branch coupled with the distal end of the first arm and a second branch coupled with the distal end of the second arm,
a hollow casing comprising an elongated tube capable of laparoscopic introduction into a body, the hollow casing having a hollow interior configured to be capable of slidably receiving the implantable device therein,
wherein the first arm, the second arm and the lift system are slidably disposed within the hollow interior of the hollow casing such that the first arm is capable of moving independently of the second arm and operation of the second arm causes the first component to become engaged with the second component;
inserting the hollow casing laparoscopically into an abdomen;
positioning the first component adjacent to a first surface of a tissue of interest through operation of the first arm of the delivery device; and
positioning the portion of the second component that is magnetically biased to attract a portion of the first component adjacent to a second surface of the tissue of interest through operation of the second arm of the delivery device such that the first component magnetically engages the second component through the tissue of interest, the at least two pins of the first component couple with the second component, and the tissue of interest is disposed therebetween.

30. The method of claim 29, wherein the first arm of the delivery device is further capable of rotational movement.

31. The method of claim 29, further comprising the steps of:
uncoupling the first arm of the delivery device from the first component of the implantable device;
uncoupling the second arm of the delivery device from the second component of the implantable device; and
withdrawing the delivery device from the body.

32. The method of claim 29, wherein at least one of the at least two pins of the first component is moveable between a substantially retracted position and a substantially extended position and further comprising the step of moving the at least one moveable pin of the first component to the substantially extended position.

33. The method of claim 32, wherein the step of positioning the portion of the second component that is magnetically biased to attract a portion of the first component adjacent to a second surface of the tissue of interest through operation of the second arm of the delivery device further comprises the steps of:
advancing the second component through the hollow casing; and
operating the second arm of the delivery device to rotate the second component such that the portion of the second component that is magnetically biased to attract a portion of the first component magnetically engages the portion of the first component through the tissue of interest.

34. The method of claim 31, wherein the distal end of the second arm of the delivery device further comprises a screw-like tip, the second component of the implantable device further comprises a hollow interior configured to receive the screw-like tip of the second arm, and the step of uncoupling the second arm of the delivery device from the second component of the implantable device further comprises the step of unscrewing the screw-like tip of the second arm from the hollow interior of the second component.

35. A kit for performing a medical procedure comprising:
an implantable device comprising a first component comprising at least two pins extending therefrom and at least one magnet, a second component comprising at least one magnet having a portion that is magnetically biased to attract a portion of the first component, and the first and second components are configured to magnetically engage one another through a targeted tissue and the creation of an attractive magnetic force between the first component and the second component causes the releasable coupling of the at least two pins of the first component with the second component, thereby defining an interior space between the first component and the second component and mechanically engaging the targeted tissue therebetween.

36. The kit of claim 35, further comprising a fluoroscope.

37. The kit of claim 35, further comprising an endoscopic camera.

38. An implantable device comprising:
a first component comprising at least one magnet;
a second component comprising at least one magnet;
a magnetic graft configured for placement within a vessel lumen, the magnetic graft comprising a proximal end, a distal end, a body and a hollow interior, both the body and the hollow interior extending between the proximal end and the distal end; and
the proximal end of the magnetic graft comprises at least one magnet having a portion that is magnetically biased to attract a portion of the first component through a first targeted tissue and a plurality of pins extending radially therefrom, the distal end of the magnetic graft comprises at least one magnet having a portion that is magnetically biased to attract a portion of the second component through a second targeted tissue and a plurality of pins extending radially therefrom, and the creation of an attractive magnetic force between the first component and the proximal end of the magnetic graft causes the releasable coupling of the plurality of pins of the proximal end with the first component and mechanically engages the first targeted tissue therebetween, and the creation of an attractive magnetic force between the second component and the distal end of the magnetic graft causes the releasable coupling of the plurality of pins of the distal end with the second component and mechanically engages the second targeted tissue therebetween.

39. The implantable device of claim 38, wherein the vessel lumen comprises an abdominal aorta, the first targeted tissue comprises a portion of the aorta proximal to an aortic aneurysm, and the second targeted tissue comprises a portion of the aorta distal to an aortic aneurysm.

40. The implantable device of claim 38, wherein the first and second components are capable of laparoscopic delivery to the first and second targeted tissues, respectively.

41. The implantable device of claim 38, wherein the magnetic graft is capable of endoscopic delivery.

42. The implantable device of claim 38, wherein the first and second components are configured in a C-shaped configuration.

43. The implantable device of claim 38, wherein the first and second components are configured in a ring-shaped configuration.

44. The implantable device of claim 38, wherein the first and second components comprise a flexible or semi-flexible material.

45. The implantable device of claim 38, wherein the first and second components each further comprise a joint.

46. The implantable device of claim 38, wherein the first component further comprises a plurality of receptacles, each of the receptacles configured to receive at least one of the plurality of pins of the proximal end of the magnetic graft.

47. The implantable device of claim 38, wherein the second component further comprises a plurality of receptacles, each of the receptacles configured to receive at least one of the plurality of pins of the distal end of the magnetic graft.

48. An implantable device comprising:
a first component comprising at least one magnet and a plurality of pins extending therefrom;
a second component comprising at least one magnet and a plurality of pins extending therefrom;
a magnetic graft configured for placement within a vessel lumen, the magnetic graft comprising a proximal end, a distal end, a body and a hollow interior, both the body and the hollow interior extending between the proximal end and the distal end; and
the proximal end of the magnetic graft comprises at least one magnet having a portion that is magnetically biased to attract a portion of the first component through a first targeted tissue, the distal end of the magnetic graft comprises at least one magnet having a portion that is magnetically biased to attract a portion of the second component through a second targeted tissue, and the creation of an attractive magnetic force between the first component and the proximal end of the magnetic graft causes the releasable coupling of the plurality of pins of the first component with the proximal end of the magnetic graft and mechanically engages the first targeted tissue therebetween, and the creation of an attractive magnetic force between the second component and the distal end of the magnetic graft causes the releasable coupling of the plurality of pins of the second component with the distal end of the magnetic graft and mechanically engages the second targeted tissue therebetween.
